(19) United States
(12) Patent Application Publication
(10) Pub. No.: US 2019/0359945 A1
(43) Pub. Date: Nov. 28, 2019

(54) METHOD FOR PRODUCING AN IMMUNOCONJUGATE

(71) Applicant: Hoffmann-La Roche Inc., Little Falls, NJ (US)

(72) Inventors: Ingo Gorr, Marktheidenfeld (DE); Oliver Popp, Penzberg (DE); Alina Schneider, München (DE)

(73) Assignee: Hoffmann-La Roche Inc., Little Falls, NJ (US)

(21) Appl. No.: 16/388,376

(22) Filed: Apr. 18, 2019

Related U.S. Application Data
(63) Continuation of application No. PCT/EP2017/076754, filed on Oct. 19, 2017.

(30) Foreign Application Priority Data
Oct. 19, 2016 (EP) 16194652.0

(51) Int. Cl. C12N 5/071; A61K 47/68; C12N 15/85

(52) U.S. Cl. CPC C12N 5/0682; A61K 47/6845; A61K 47/6849; C12N 2015/8518; C12N 2523/00; C12N 2500/60; C12N 15/85

(57) ABSTRACT

Herein is reported a method for producing an immunoconjugate with reduced product- and process-related impurities by culturing mammalian cells that contain one or more nucleic acids encoding the immunoconjugate of interest in a cell culture medium, wherein the one or more nucleic acids are expressed under the conditions of cell culture comprising the steps of:
culturing the mammalian cells in a cell culture medium at a first temperature and at a first pH;
reducing the first temperature of the cell culture medium to a second temperature; and increasing the first pH of the cell culture medium to a second pH;
recovering the immunoconjugate from the cells or the cell culture medium,
   and thereby producing the immunoconjugate.

### CROSS REFERENCE TO RELATED APPLICATIONS

This application is a continuation of International Application No. PCT/EP2017/076754 having an International Filing Date of 19 Oct. 2017, claiming priority to application number EP 16194652.0 filed 19 Oct. 2016, each of which are incorporated herein by reference in its entirety.

### SEQUENCE LISTING

This application contains a Sequence Listing which has been submitted electronically in ASCII format and is hereby incorporated by reference in its entirety. Said ASCII copy, created on Mar. 13, 2019, is named P33923-US_Sequence_Listing.txt and is 38,523 bytes in size.

### FIELD OF THE INVENTION

The current invention is in the field of polypeptide production and fermentation processes. Herein is reported a novel method for the production of immunoconjugates.

### BACKGROUND OF THE INVENTION

Cancer is one of the most abundant causes of death worldwide. In 2012, 8.2 million deaths were registered; mostly caused by lung, liver, stomach, colorectal and breast cancer. The conservative cancer therapy consists of one or a combination of the following treatments: surgery, radiotherapy, and chemotherapy (WHO 2014). Another possibility for cancer treatment is immunotherapy, spurring the body's own immune response to cancer. Cytokines are cell signaling molecules that participate in regulation of the immune system. When used in cancer therapy, cytokines can act as immunomodulatory agents that have anti-tumor effects and which can increase the immunogenicity of some types of tumors. In this therapeutic area Interleukin-2 (IL-2) was the first drug to be effective against human cancer. IL-2 is a potent molecule in cancer therapy. IL-2 is a cytokine, which plays a crucial role in the immune response. It is produced in T helper (Th, CD4+) cells and, to a lesser amount, in other cells of the immune system, including CD8+ T cells. Clinical application of Proleukin® (aldesleukin), the commercially available form of IL-2, however, is limited due to the high toxicity of the molecule during treatment. IL-2 immunoconjugates have been developed to circumvent the drawbacks of the current IL-2 therapy. These molecules may target a tumor marker (TM) and, hence, realizes a local effect of IL-2 in TM bearing tumor tissue or IL-2 may be attached to untargeted IgG molecules leading e.g. to extended half-life. In addition, IL-2 itself and the Fc region can be modified for decreased toxicity of the molecule (IL-2 variants). Nevertheless, during production of the IL-2 immunoconjugate in CHO cells different product related and process related impurities are observed. These are e.g. molecules with fragmented cytokine (IL-2) moieties, aggregated molecules (high molecular weights species; HMWs) or host cell proteins like Clusterin or phospholipase B-like 2 (PLBL2/PLB2). Fragmentation occurs only in the IL-2 molecule, whereas no clipping is observed in the antibody. According to FDA regulations the product has to be devoid of any heterogeneities including aggregation, denaturation and fragmentation (Zoon, K. C. 1997 “Points to Consider in the Manufacture and Testing of Monoclonal Antibody Products for Human Use”, U. S. Department of Health and Human Services, Food and Drug Administration). To ensure the safety of biopharmaceutical agents to humans by-products accumulating during the production process have to be removed and it is desired to prevent the formation and accumulation of these impurities from the outset.

In WO2008/131374 a method for reduction of protein misfolding and aggregation in the cell culture by growing the cell culture at a reduced temperature and/or reduced pH is described.

Cruz et al. (Biotechnology Letters, pages 677-682, (2000)) report on product quality of a recombinant fusion protein expressed in immobilised baby hamster kidney cells grown in protein-free medium.

In WO 2012/146628 antigen-specific immunoconjugates for selectively delivering effector moieties that influence cellular activity are reported.

Trummer et al. (Biotechnol Bioeng, 2006, 94(6): 1045-1052) describe a biphasic cultivation as a tool for enhancing the volumetric productivity of batch processes using Epo-Fc expressing CHO cells.

An improved cell cultivation process is reported in WO 2011/134919.

### SUMMARY OF THE INVENTION

Herein is reported a method for producing an immunoconjugate with reduced levels of product related impurities as well as process related impurities. In more detail, it has been found that the occurrence of fragments of immunoconjugates (e.g. fragmented cytokine such as IL-2), host cell proteins (such as, e.g. PLBL2 or Clusterin) and aggregates in the cultivation medium can be reduced by growing the cell culture/cultivating the cells at a first (higher) temperature and a first, (lower) pH during a first period of time and then shifting the temperature to a lower value and the pH to a higher value until the end of the cultivation.

Accordingly, one aspect as reported herein is a method for producing an immunoconjugate with reduced product- and process-related impurities by culturing mammalian cells that contain one or more nucleic acids encoding the immunoconjugate of interest in a cell culture medium, wherein the one or more nucleic acids are expressed under the conditions of cell culture comprising the steps of:
culturing the mammalian cells in a cell culture medium at a first temperature and at a first pH;
reducing the first temperature of the cell culture medium to a second temperature; and increasing the first pH of the cell culture medium to a second pH;
recovering the immunoconjugate from the cells or the cell culture medium,
   and thereby producing the immunoconjugate.

In one embodiment the method further comprises purifying the immunoconjugate with one or more purification steps.

In one embodiment the reduction to the second temperature and the increase to a second pH is (performed) after 2 to 9 days of a total cultivation time of 13 to 14 days. In one embodiment the reduction to the second temperature and the increase to a second pH is (performed) after about 8 days of a total cultivation time of 13 to 14 days.

In one embodiment the reduction to the second temperature and the increase to a second pH is (performed) at about the same time.

In one embodiment the first temperature is 37° C.+/−0.5° C. and the second temperature is in the range of 28° C. to 34° C. (i.e. the first temperature is reduced by 3° C. to 9° C.). In one embodiment the first temperature is 37° C.+/−0.5° C. and the second temperature is in the range of 29° C. to 30° C. (i.e. is reduced by 7° C. to 8° C.). In one embodiment the first temperature is 37° C.+/−0.5° C. and is reduced to a second temperature of 29° C.+/−0.5° C.

In one embodiment the second pH is 7.25 or higher.

In one embodiment the inoculation cell density is from about 600000 (6×10⁵) to about 1000000 (1×10⁶) cells/ml. In one embodiment the inoculation cell density is about 600000 (6×10⁵) or about 1000000 (1×10⁶) cells/ml.

In one embodiment the osmolality of the cell culture medium is 300 mOsmol/kg+/−15 mOsmol/kg or more. In one embodiment the osmolality of the cell culture medium is 350 mOsmol/kg+/−15 mOsmol/kg or more. In one embodiment the osmolality of the cell culture medium is from 350 mOsmol/kg+/−15 mOsmol/kg to 800 mOsmol/kg+/−15 mOsmol/kg. In one embodiment the osmolality of the cell culture medium is about 400 mOsmol/kg.

In one embodiment the immunoconjugate is a CEA-targeted IL-2 immunoconjugate, a FAP-targeted IL-2 immunoconjugate or an IgG-IL-2 immunoconjugate or a FAP-targeted 4-1-BBL immunoconjugate.

One aspect as reported herein is an immunoconjugate obtainable with the method as reported herein.

One aspect as reported herein is a composition comprising an immunoconjugate and a reduced level of fragments (such as fragmented IL-2), aggregates and host cell proteins.

In one embodiment the immunoconjugate obtainable with the method or in the composition as reported herein is a CEA-targeted IL-2 immunoconjugate, a FAP-targeted IL-2 immunoconjugate or an IgG-IL-2 immunoconjugate or a FAP-targeted 4-1-BBL immunoconjugate.

In one embodiment the cell culture is a large-scale cell culture.

In one embodiment the cell culture has a volume of about 100 liters to about 15000 liters.

In one embodiment the cell culture is a batch cell culture.

In one embodiment the cell culture is a fed-batch cell culture.

In one embodiment the medium is a serum free medium.

In one embodiment the medium is a protein-free medium.

In one embodiment the medium is a chemically-defined medium.

In one embodiment the medium is a protein-free chemically defined medium.

In one embodiment the mammalian cell is a CHO cell.

### DETAILED DESCRIPTION OF THE INVENTION

### Terminology

As used herein, the term “immunoconjugate” refers to a fusion polypeptide molecule that includes at least one effector moiety and at least one immunoglobulin moiety. In certain embodiments, the immunoconjugate comprises one effector moiety and one immunoglobulin moiety. In certain embodiments, the immunoconjugate comprises one effector moiety and one IgG immunoglobulin moiety. In certain embodiments, the immunoconjugate comprises two effector moieties and one immunoglobulin moiety. An immunoconjugate as referred to herein, is a fusion protein, i.e. the components of the immunoconjugate are linked to each other by peptide-bonds, either directly or through one or more linker peptides. Particular immunoconjugates according to the invention essentially consist of at least one effector moiety and one immunoglobulin moiety, joined by one or more linker sequences.

The term “immunoglobulin moiety” refers to a protein having the structure of a naturally occurring antibody. For example, immunoglobulins of the IgG class are heterotetrameric glycoproteins of about 150,000 daltons, composed of two light chains and two heavy chains that are disulfide-bonded to each other. From N- to C-terminus, each heavy chain has a variable region (VH), also called a variable heavy domain or a heavy chain variable domain, followed by three constant domains (CH1, CH2, and CH3), also called a heavy chain constant region, whereby between the first and the second constant domain a hinge region is located. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five types, called a (IgA), δ (IgD), ε (IgE), γ (IgG), or μ (IgM), some of which may be further divided into subtypes, e.g. γ1 (IgG), γ2 (IgG2), γ3 (IgG3), γ4 (IgG4), α1 (IgA1) and α2 (IgA2). The light chain of an immunoglobulin may be assigned to one of two types, called kappa (κ) and lambda (λ), based on the amino acid sequence of its constant domain. The immunoglobulin moiety may be a polypeptide molecule that specifically binds to an antigenic determinant. In one embodiment, an immunoglobulin moiety is able to direct the entity to which it is attached to a target site, for example to a specific type of tumor cell or tumor stroma bearing the antigenic determinant. Immunoglobulin moieties include antibodies and antibody fragments.

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an immunoglobulin moiety binds. Useful antigenic determinants can be found, for example, on the surfaces of tumor cells, on the surfaces of virus-infected cells, on the surfaces of other diseased cells, free in blood serum, and/or in the extracellular matrix (ECM). In a particular embodiment the antigenic determinant is a human antigen.

In certain embodiments, non-limiting examples of tumor antigens include MAGE, MART-1/Melan-A, gp 100, Dipeptidyl peptidase IV (DPPIV), adenosine deaminase-binding protein (ADAbp), cyclophilin b, Colorectal associated antigen (CRC)-C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117 PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2.

In certain embodiments, non-limiting examples of viral antigens include influenza virus hemagglutinin, Epstein-Barr virus LMP-1, hepatitis C virus E2 glycoprotein, HIV gp160, and HIV gp120.

In certain embodiments, non-limiting examples of ECM antigens include syndecan, heparanase, integrins, osteopontin, link, cadherins, laminin, laminin type EGF, lectin, fibronectin, notch, tenascin, and matrixin.

In certain embodiments, the immunoconjugates described herein can bind to the following specific non-limiting examples of cell surface antigens: FAP (Fibroblast Activation Protein), Her2, EGFR, IGF-1R, CD2 (T-cell surface antigen), CD3 (heteromultimer associated with the TCR), CD22 (B-cell receptor), CD23 (low affinity IgE receptor), CD30 (cytokine receptor), CD33 (myeloid cell surface antigen), CD40 (tumor necrosis factor receptor), IL-6R (IL6 receptor), CD20, MCSP, and PDGFβR (β platelet-derived growth factor receptor).

The invention provides not only immunoconjugates targeted to a specific antigen (e.g. a tumor antigen) but also untargeted conjugates comprising one or more immunoglobulin moieties which do not specifically bind to any antigen, particularly not bind to any human antigen. The absence of specific binding of these conjugates to any antigen (i.e. the absence of any binding that can be discriminated from non-specific interaction) can be measured e.g. by ELISA or surface plasmon resonance as described herein. Such conjugates are particularly useful e.g. for enhancing the serum half-life of the effector moiety they comprise, as compared to the serum half-life of the unconjugated effector moiety, where targeting to a particular tissue is not desired. An example of an untargeted immunoconjugate is IgG-IL-2. A targeted immunoconjugate produced by the method according to the invention denotes a conjugate of an antibody specifically binding to the target (antigen) and an effector moiety such as e.g. a cytokine. Thus, a FAP targeted IL-2 immunoconjugate denotes an antibody binding to FAP conjugated to IL-2.

As used herein, the term “effector moiety” refers to a polypeptide, e.g., a protein or glycoprotein, that influences cellular activity, for example, through signal transduction or other cellular pathways. Accordingly, the effector moiety of the invention can be associated with receptor-mediated signaling that transmits a signal from outside the cell membrane to modulate a response in a cell bearing one or more receptors for the effector moiety. In one embodiment, an effector moiety can elicit a cytotoxic response in cells bearing one or more receptors for the effector moiety. In another embodiment, an effector moiety can elicit a proliferative response in cells bearing one or more receptors for the effector moiety. In another embodiment, an effector moiety can elicit differentiation in cells bearing receptors for the effector moiety. In another embodiment, an effector moiety can alter expression (i.e. upregulate or downregulate) of an endogenous cellular protein in cells bearing receptors for the effector moiety. Non-limiting examples of effector moieties include cytokines, growth factors, hormones, enzymes, substrates, and cofactors. In one embodiment, the effector moiety is a cytokine. In one embodiment, the effector moiety is an interleukin. In one embodiment, the effector moiety is an interleukin 2 (IL-2). In one embodiment, the effector moiety is a 4-1BB-ligand (4-1BBL).

As used herein, the term “cytokine” refers to a molecule that mediates and/or regulates a biological or cellular function or process (e.g. immunity, inflammation, and hematopoiesis). The term “cytokine” as used herein includes “lymphokines,” “chemokines,” “monokines,” and “interleukins”. In certain embodiments, examples of useful cytokines include, but are not limited to, GM-CSF, IL-1α, IL-1β, IL-2, IL-3, IL-4, IL-5, IL-6, IL-7, IL-8, IL-10, IL-12, 4-1BB-ligand (4-1BBL), IFN-α, IFN-β, IFN-γ, MIP-1α, MIP-1β, TGF-β, TNF-α, and TNF-β. Particular cytokines are IL-2, IL-7, IL-10, IL-12, IL-15, IFN-α and IFN-γ. In particular embodiments the cytokine is a human cytokine. The term “cytokine” as used herein is meant to also include cytokine variants comprising one or more amino acid mutations in the amino acid sequences of the corresponding wild-type cytokine, such as for example the IL-2 variants described in Sauvé et al., Proc Natl Acad Sci USA 88, 4636-40 (1991); Hu et al., Blood 101, 4853-4861 (2003) and US Pat. Publ. No. 2003/0124678; Shanafelt et al., Nature Biotechnol 18, 1197-1202 (2000); Heaton et al., Cancer Res 53, 2597-602 (1993) and U.S. Pat. No. 5,229,109; US Pat. Publ. No. 2007/0036752; WO 2008/0034473; WO 2009/061853; or WO/2012/107417.

As used herein, the term “single-chain” refers to a molecule comprising amino acid monomers linearly linked by peptide bonds. In one embodiment, the effector moiety is a single-chain effector moiety. Non-limiting examples of single-chain effector moieties include cytokines, growth factors, hormones, enzymes, substrates, and cofactors. When the effector moiety is a cytokine and the cytokine of interest is normally found as a multimer in nature, each subunit of the multimeric cytokine is sequentially encoded by the single-chain of the effector moiety. Accordingly, non-limiting examples of useful single-chain effector moieties include GM-CSF, IL-1α, IL-1β, IL-2, IL-3, IL-4, IL-5, IL-6, IL-7, IL-8, IL-10, IL-12, IL-15, 4-1BB-ligand (4-1BBL), IFN-α, IFN-β, IFN-γ, MIP-1α, MIP-1β, TGF-β, TNF-α, and TNF-β.

As used herein, the term “effector moiety receptor” refers to a polypeptide molecule capable of binding specifically to an effector moiety. For example, where IL-2 is the effector moiety, the effector moiety receptor that binds to an IL-2 molecule (e.g. an immunoconjugate comprising IL-2) is the IL-2 receptor. Where an effector moiety specifically binds to more than one receptor, all receptors that specifically bind to the effector moiety are “effector moiety receptors” for that effector moiety.

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, Md. (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, Md. (1991) is used for the light chain constant domain CL of kappa and lambda isotype. Specifically the Kabat EU index numbering system (see pages 661-723) is used for the constant heavy chain domains (CH1, Hinge, CH2 and CH3, which is herein further clarified by referring to “numbering according to Kabat EU index” in this case).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

To a person skilled in the art procedures and methods are well known to convert an amino acid sequence, e.g. of a polypeptide, into a corresponding nucleic acid sequence encoding this amino acid sequence. Therefore, a nucleic acid is characterized by its nucleic acid sequence consisting of individual nucleotides and likewise by the amino acid sequence of a polypeptide encoded thereby.

The term “about” denotes a range of +/−20% of the thereafter following numerical value. In one embodiment the term about denotes a range of +/−10% of the thereafter following numerical value. In one embodiment the term about denotes a range of +/−5% of the thereafter following numerical value.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term “antibody-dependent cellular cytotoxicity (ADCC)” is a function mediated by Fc receptor binding and refers to lysis of target cells by an antibody as reported herein in the presence of effector cells. ADCC is measured in one embodiment by the treatment of a preparation of CD19 expressing erythroid cells (e.g. K562 cells expressing recombinant human CD19) with an antibody as reported herein in the presence of effector cells such as freshly isolated PBMC (peripheral blood mononuclear cells) or purified effector cells from buffy coats, like monocytes or NK (natural killer) cells. Target cells are labeled with Cr-51 and subsequently incubated with the antibody. The labeled cells are incubated with effector cells and the supernatant is analyzed for released Cr-51. Controls include the incubation of the target endothelial cells with effector cells but without the antibody. The capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA). In one preferred embodiment binding to FcγR on NK cells is measured.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term “complement-dependent cytotoxicity (CDC)” refers to lysis of cells induced by the antibody as reported herein in the presence of complement. CDC is measured in one embodiment by the treatment of CD19 expressing human endothelial cells with an antibody as reported herein in the presence of complement. The cells are in one embodiment labeled with calcein. CDC is found if the antibody induces lysis of 20% or more of the target cells at a concentration of 30 μg/ml. Binding to the complement factor C1q can be measured in an ELISA.

In such an assay in principle an ELISA plate is coated with concentration ranges of the antibody, to which purified human C1q or human serum is added. C1q binding is detected by an antibody directed against C1q followed by a peroxidase-labeled conjugate. Detection of binding (maximal binding Bmax) is measured as optical density at 405 nm (OD405) for peroxidase substrate ABTS® (2,2′-azino-di-[3-ethylbenzthiazoline-6-sulfonate]).

“Effector functions” refer to those biological activities attributable to the Fc region of an antibody, which vary with the antibody class. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492; Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 10³-fold and eliminated the human monocyte response to antibody-sensitized red blood cells (Armour, K. L., et al., Eur. J. Immunol. 29 (1999) 2613-2624).
FcγRII (CD32) binds complexed IgG with medium to low affinity and is widely expressed. This receptor can be divided into two sub-types, FcγRIIA and FcγRIIB. FcγRIIA is found on many cells involved in killing (e.g. macrophages, monocytes, neutrophils) and seems able to activate the killing process. FcγRIIB seems to play a role in inhibitory processes and is found on B cells, macrophages and on mast cells and eosinophils. On B-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat).
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

The term “Fc receptor” as used herein refers to activation receptors characterized by the presence of a cytoplasmatic ITAM sequence associated with the receptor (see e.g. Ravetch, J. V. and Bolland, S., Annu. Rev. Immunol. 19 (2001) 275-290). Such receptors are FcγRI, FcγRIIA and FcγRIIIA. The term “no binding of FcγR” denotes that at an antibody concentration of 10 μg/ml the binding of an antibody as reported herein to NK cells is 10% or less of the binding found for anti-OX40L antibody LC.001 as reported in WO 2006/029879.

While IgG4 shows reduced FcR binding, antibodies of other IgG subclasses show strong binding. However Pro238, Asp265, Asp270, Asn297 (loss of Fc carbohydrate), Pro329 and 234, 235, 236 and 237 Ile253, Ser254, Lys288, Thr307, Gln311, Asn434, and His435 are residues which provide if altered also reduce FcR binding (Shields, R. L., et al. J. Biol. Chem. 276 (2001) 6591-6604; Lund, J., et al., FASEB J. 9 (1995) 115-119; Morgan, A., et al., Immunology 86 (1995) 319-324; and EP 0 307 434).

The term “Fc-region” herein is used to define a C-terminal region of an immunoglobulin heavy chain that contains at least a portion of the constant region. The term includes native sequence Fc-regions and variant Fc-regions. In one embodiment, a human IgG heavy chain Fc-region extends from Cys226, or from Pro230, or from Ala 231 to the carboxyl-terminus of the heavy chain. However, the C-terminal lysine (Lys447) of the Fc region may or may not be present.

The antibody molecules of the conjugates as reported herein comprise as Fc-region, in one embodiment an Fc-region derived from human origin. In one embodiment the Fc-region comprises all parts of the human constant region. The Fc-region of an antibody is directly involved in complement activation, C1q binding, C3 activation and Fc receptor binding. While the influence of an antibody on the complement system is dependent on certain conditions, binding to C1q is caused by defined binding sites in the Fc-region. Such binding sites are known in the state of the art and described e.g. by Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virol. 75 (2001) 12161-12168; Morgan, A., et al., Immunology 86 (1995) 319-324; and EP 0 307 434. Such binding sites are e.g. L234, L235, D270, N297, E318, K320, K322, P331 and P329 (numbering according to EU index of Kabat). Antibodies of subclass IgG1, IgG2 and IgG3 usually show complement activation, C1q binding and C3 activation, whereas IgG4 do not activate the complement system, do not bind C1q and do not activate C3. An “Fc-region of an antibody” is a term well known to the skilled artisan and defined on the basis of papain cleavage of antibodies. In one embodiment the Fc-region is a human Fc-region. In one embodiment the Fc-region is of the human IgG4 subclass comprising the mutations S228P and/or L235E and/or P329G (numbering according to EU index of Kabat). In one embodiment the Fc-region is of the human IgG1 subclass comprising the mutations L234A and L235A and optionally P329G (numbering according to EU index of Kabat).

The term “wild-type Fc-region” denotes an amino acid sequence identical to the amino acid sequence of an Fc-region found in nature. Wild-type human Fc-regions include a native human IgG1 Fc-region (non-A and A allotypes), native human IgG2 Fc-region, native human IgG3 Fc-region, and native human IgG4 Fc-region as well as naturally occurring variants thereof. Wild-type Fc-regions are denoted in SEQ ID NO: 26 (IgG1, caucasian allotype), SEQ ID NO: 27 (IgG1, afroamerican allotype), SEQ ID NO: 28 (IgG2), SEQ ID NO: 29 (IgG3) and SEQ ID NO: 30 (IgG4).

Variant (human) Fc-regions are defined by the amino acid mutations that are contained. Thus, for example, the term P329G denotes a variant Fc-region with the mutation of proline to glycine at amino acid position 329 relative to the parent (wild-type) Fc-region (numbering according to EU index of Kabat). The identity of the wild-type amino acid may be unspecified, in which case the aforementioned variant is referred to as 329G.

The terms “full length antibody”, “intact antibody,” and “whole antibody” are used herein interchangeably to refer to an antibody having a structure substantially similar to a native antibody structure or having heavy chains that contain an Fc region as defined herein.

The term “hinge region” denotes the part of an antibody heavy chain polypeptide that joins in a wild-type antibody heavy chain the CH1 domain and the CH2 domain, e. g. from about position 216 to about position 230 according to the EU number system of Kabat, or from about position 226 to about position 230 according to the EU number system of Kabat. The hinge regions of other IgG subclasses can be determined by aligning with the hinge-region cysteine residues of the IgG1 subclass sequence.

The hinge region is normally a dimeric molecule consisting of two polypeptides with identical amino acid sequence. The hinge region generally comprises about 25 amino acid residues and is flexible allowing the associated target binding sites to move independently. The hinge region can be subdivided into three domains: the upper, the middle, and the lower hinge domain (see e.g. Roux, et al., J. Immunol. 161 (1998) 4083).

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR”, as used herein, refers to each of the regions of an antibody variable domain comprising the amino acid residue stretches which are hypervariable in sequence (“complementarity determining regions” or “CDRs”) and/or form structurally defined loops (“hypervariable loops”), and/or contain the antigen-contacting residues (“antigen contacts”). Generally, antibodies comprise six HVRs; three in the VH (H1, H2, H3), and three in the VL (L, L2, L3).

HVRs include
(a) hypervariable loops occurring at amino acid residues 26-32 (L), 50-52 (L2), 91-96 (L3), 26-32 (H1), 53-55 (H2), and 96-101 (H3) (Chothia, C. and Lesk, A. M., J. Mol. Biol. 196 (1987) 901-917);
(b) CDRs occurring at amino acid residues 24-34 (L), 50-56 (L2), 89-97 (L3), 31-35b (H1), 50-65 (H2), and 95-102 (H3) (Kabat, E. A. et al., Sequences of Proteins of Immunological Interest, 5th ed. Public Health Service, National Institutes of Health, Bethesda, Md. (1991), NIH Publication 91-3242.);
(c) antigen contacts occurring at amino acid residues 27c-36 (L), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “isolated” antibody is one, which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman, S. et al., J. Chromatogr. B 848 (2007) 79-87.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “light chain” denotes the shorter polypeptide chains of native IgG antibodies. The light chain of an antibody may be assigned to one of two types, called kappa (κ) and lambda (λ), based on the amino acid sequence of its constant domain.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt, T. J. et al. Kuby Immunology, 6th ed., W.H. Freeman and Co., N.Y. (2007), page 91) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano, S. et al., J. Immunol. 150 (1993) 880-887; Clackson, T. et al., Nature 352 (1991) 624-628).

### Purification of Antibodies and Immunoconjugates

The term “affinity chromatography” as used within this application denotes a chromatography method which employs an “affinity chromatography material”. In an affinity chromatography antibodies are separated based on their biological activity or chemical structure depending on the formation of electrostatic interactions, hydrophobic bonds, and/or hydrogen bonds to the chromatographical functional groups of the chromatography material. To recover the specifically bound antibody from the affinity chromatography material either a competitor ligand can be added or the chromatography conditions, such as pH value, polarity or ionic strength of the buffer, can be changed. Exemplary “affinity chromatography materials” are metal chelating chromatography materials such as Ni(II)—NTA or Cu(II)—NTA, or antibody affinity chromatography materials such as chromatography materials comprising thereto covalently linked protein A or protein G, or enzyme binding affinity chromatography materials such as chromatography materials comprising thereto covalently bound enzyme substrate analogues, enzyme cofactors, or enzyme inhibitors as chromatographical functional group, or lectin binding chromatography materials such as chromatography materials comprising thereto covalently linked polysaccharides, cell surface receptors, glycoproteins, or intact cells as chromatographical functional group.

In one embodiment the antibody light chain affinity ligand uses a light chain constant domain specific capture reagent, which e.g. specific for the kappa or the lambda constant light chain, depending on whether a kappa or a lambda light chain is contained in the antibody. Examples of such light chain constant domain specific capture reagents are e.g. KappaSelect™ and LambdaFabSelect™ (available from GE Healthcare/BAC), which are based on a highly rigid agarose base matrix that allows high flow rates and low back pressure at large scale. These materials contain a ligand that binds to the constant region of the kappa or the lambda light chain, respectively (antibodies or fragments thereof lacking the constant region of the light chain will not bind). Both are therefore capable of binding other target molecules containing the constant region of the light chain, for example, IgG, IgA and IgM. The ligands are attached to the matrix via a long hydrophilic spacer arm to make them easily available for binding to the target molecule. They are based on a single-chain antibody fragment that is screened for either human Ig kappa or lambda.

The term “applying to” and grammatical equivalents thereof as used within this application denotes a partial step of a purification method in which a solution containing a substance of interest is brought in contact with a stationary phase. The solution containing the substance of interest to be purified passes through the stationary phase providing for an interaction between the stationary phase and the substances in solution. Depending on the conditions, such as e.g. pH, conductivity, salt concentration, temperature, and/or flow rate, some substances of the solution are bound to the stationary phase and therewith are removed from the solution. Other substances remain in solution. The substances remaining in solution can be found in the flow-through. The “flow-through” denotes the solution obtained after the passage of the chromatographic device, which may either be the applied solution containing the substance of interest or the buffer, which is used to flush the column or to cause elution of one or more substances bound to the stationary phase. The substance of interest can be recovered from the solution after the purification step by methods familiar to a person of skill in the art, such as e.g. precipitation, salting out, ultrafiltration, diafiltration, lyophilization, affinity chromatography, or solvent volume reduction to obtain the substance in substantially homogeneous form.

An antibody or antibody fragment or immunoconjugate can be produced by recombinant means. Methods for recombinant production are widely known in the state of the art and comprise protein expression in eukaryotic cells with subsequent isolation of the antibody or antibody fragment or immunoconjugate and purification to a pharmaceutically acceptable purity. For the expression of the antibody or antibody fragment or immunoconjugate either a hybridoma cell or a eukaryotic cell, in which one or more nucleic acids encoding the antibody or antibody fragment have been introduced, is used. In one embodiment the eukaryotic cells is selected from CHO cells (e.g. CHO K1, CHO DG44), NSO cells, SP2/0 cells, HEK 293 cells, COS cells, PER.C6 cells, BHK cells, rabbit cells, or sheep cells. In another embodiment the eukaryotic cell is selected from CHO cells, HEK cells, or rabbit cells. After expression the antibody or antibody fragment or immunoconjugate is recovered from the cells (from the supernatant or from the cells after lysis). General methods for recombinant production of antibodies are well-known in the state of the art and reported, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-160; Werner, R. G., Drug Res. 48 (1998) 870-880.

Purification of antibodies or immunoconjugates can be performed in order to eliminate cellular components or other contaminants, e.g. other cellular nucleic acids or proteins, by standard techniques, including alkaline/SDS treatment, CsCl banding, column chromatography, agarose gel electrophoresis, and others well known in the art (see e.g. Ausubel, F. M, et al. (eds.), Current Protocols in Molecular Biology, John Wiley & Sons, Inc., New York (2005)). Different methods are well established and widespread used for protein purification, such as affinity chromatography with microbial proteins (e.g. protein A or protein G affinity chromatography), ion exchange chromatography (e.g. cation exchange (carboxymethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis), as well as combinations thereof, such as affinity chromatography with microbial proteins, cation exchange chromatography and anion exchange chromatography (see e.g. Vijayalakshmi, M. A., Appl. Biochem. Biotech. 75 (1998) 93-102). General chromatographic methods and their use are known to a person skilled in the art. See for example, Heftmann, E. (ed.), Chromatography, 5^{th} edition, Part A: Fundamentals and Techniques, Elsevier Science Publishing Company, New York (1992); Deyl, Z. (ed.), Advanced Chromatographic and Electromigration Methods in Biosciences, Elsevier Science BV, Amsterdam, The Netherlands (1998); Poole, C. F., and Poole, S. K., Chromatography Today, Elsevier Science Publishing Company, New York (1991); Scopes, Protein Purification: Principles and Practice (1982); Sambrook, J., et al. (eds.), Molecular Cloning: A Laboratory Manual, Second Edition, Cold Spring Harbor Laboratory Press, Cold Spring Harbor, N.Y. (1989); or Ausubel, F. M., et al. (eds.), Current Protocols in Molecular Biology, John Wiley & Sons, Inc., New York (2005).

For the purification of antibodies or antibody fragments or immunoconjugates, which have been produced e.g. by cell cultivation methods, generally a combination of different chromatography steps can be employed. Normally a (protein A) affinity chromatography is followed by one or two additional separation steps. In one embodiment the additional chromatography steps are a cation and an anion exchange chromatography step or vice versa. The final purification step is a so called “polishing step” for the removal of trace impurities and contaminants like aggregated immunoglobulins, residual HCP (host cell protein), DNA (host cell nucleic acid), viruses, or endotoxins.

It is understood that the molecules/immunoconjugates produced by the methods as reported herein and the compositions comprising the molecules/immunoconjugates can be subject to subsequent treatment like purification, filtration etc. which lead to further quality improvement and reduction of product- and process-related impurities.

### Cultivation of Cells

“Biomass” as used herein refers to the quantity or weight of cultured cells in the culture medium. Biomass may be measured directly or indirectly by determining viable cell density, total cell density, cell time integral (for viable and total cell density), cell volume time integral (for viable and total cell density), packed cell volume, dry weight or wet weight.

“Bioreactor” as used herein refers to any vessel used for the growth of a mammalian cell culture. Volumes of a bioreactor may vary greatly from only few millilitres to 12,000 litres or more. The internal conditions of the bioreactor, including but not limited to pH, dissolved oxygen and temperature, are typically controlled during the culture period. A bioreactor can be composed of any material that is suitable for holding mammalian cell cultures suspended in media under the culture conditions of the present invention, including glass, plastic or metal.

The terms “cell line”, “host cell”, “host cell line”, and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. The term “cell” includes cells which are used for the expression of nucleic acids. In one embodiment the host cell is a CHO cell (e.g. CHO K1, CHO DG44), or a BHK cell, or a NSO cell, or a SP2/0 cell, or a HEK 293 cell, or a HEK 293 EBNA cell, or a PER.C6® cell, or a COS cells. In another embodiment the cell is a CHO cell, or a BHK cell, or a PER.C6® cell. As used herein, the expression “cell” includes the subject cell and its progeny.

“Cell density” as used herein refers to the number of cells present in a given volume of medium.

“Inoculation cell density” or “inoculation density” as used herein refers to the cell density present at the beginning of a culture process.

“Cell viability” as used herein refers to the ability of cells in culture to survive under a given set of culture conditions or experimental variations. The term as used herein also refers to that portion of cells which are alive at a particular time in relation to the total number of cells, living or dead, in culture at that time.

“Culture” or “cell culture” as used herein refers to a cell population that is suspended in a medium under conditions suitable for survival and/or growth of the cell population. These terms will also be applied to the combination of the medium and cell population suspended therein.

“Culturing” a cell refers to contacting a cell with a cell culture medium under conditions suitable to the viability and/or growth and/or proliferation of the cell.

“Culture conditions”, “cultivation conditions” and “fermentation conditions” are used herein interchangeably and are those conditions that must be satisfied to achieve successful cell culture. Typically these conditions include provision of an appropriate medium, as well as control of e.g. temperature, which should be about 37° C., but could also include a temperature shift during culture (e.g. 37° C. to 29° C.) and pH, which is typically between 6.8 and 7.2, as well as the provision of oxygen and carbon dioxide. Such conditions also include the manner in which the cells are cultured, e.g. shaker or robotic cultivation.

The terms “medium” and “cell culture medium” refer to a nutrient source used for growing or maintaining cells. As is understood by a person of skill in the art, the nutrient source may contain components required by the cell for growth and/or survival and/or product generation, or may contain components that aid in cell growth and/or survival and/or product generation. Vitamins, essential or non-essential amino acids, trace elements, and surfactants (e.g., poloxamers) are examples of medium components. Typically such solutions containing nutrients provide essential and non-essential amino acids, vitamins, energy sources, lipids and trace elements required by the cell for minimal growth and/or survival. Such a solution may also contain supplementary components that enhance growth and/or survival above the minimal rate including, but not limited to, hormones and/or other growth factors, particular ions, such as sodium, chloride, calcium, magnesium and phosphate, buffers, vitamins, nucleosides or nucleotides, trace elements, amino acids, lipids and/or glucose or other energy source. A medium is advantageously formulated to a pH and salt concentration optimal for cell survival and proliferation. A medium may be a reduced serum or serum free medium, i.e. wherein the medium contains about 1-5% serum or when the medium is essentially free of any mammalian serum (e.g. fetal bovine serum), respectively. By essentially free is meant that the medium comprises between 0-5% serum, preferably between about 0-1% serum and most preferably about 0-0.1% serum. A serum-free defined medium may be used, where the identity and concentration of each of the components of the medium is known. A medium may be a protein-free medium, i.e. this will contain no protein but will contain undefined peptides e.g. from plant hydrolysates Media could include human serum albumin and human transferrin but potentially animal-derived insulin and lipids, or a xeno-free medium containing human serum albumin, human transferrin, human insulin and chemically defined lipids. Alternatively, a medium may be a chemically-defined medium, that is a medium wherein all substances are defined and present in defined concentrations. These media could contain only recombinant proteins and/or hormones or a protein-free chemically defined medium, i.e. containing only low molecular weight constituents and synthetic peptides/hormones if required. Chemically defined media could also be completely free of any protein. In one embodiment the medium is a serum free medium. In one embodiment the medium is a protein-free medium. In one embodiment the medium is a chemically-defined medium. In one embodiment the medium is a protein-free chemically defined medium.

For example, a “chemically defined cell culture medium” or “CDM” refers to a medium with a specified composition that is free of animal-derived products such as animal serum and peptone. The term also encompass a medium with a specified composition that is free of undefined or partially defined components, for example, components such as animal serum, an animal peptone (hydrolysate), a plant peptone (hydrolysate), and a yeast peptone (hydrolysate). As would be understood by a person of skill in the art, a CDM may be used in a process of polypeptide production whereby a cell is in contact with, and secretes a polypeptide into, the CDM. Thus, it is understood that a composition may contain a CDM and a polypeptide product and that the presence of the polypeptide product does not render the CDM chemically undefined.

A “chemically undefined cell culture medium” refers to a medium whose chemical composition cannot be specified and which may contain one or more animal-derived products such as serum and peptone. As would be understood by a person of skill in the art, a chemically undefined cell culture medium may contain an animal-derived product as a nutrient source. The term can also encompass a cell culture medium comprising undefined or partially defined components, for example, components such as an animal serum, an animal peptone (hydrolysate), a plant peptone (hydrolysate), or a yeast peptone (hydrolysate).

As used herein, “basal medium” or “base medium” refers to cell culture medium containing cell culture nutrients supplied to a culturing vessel at the start of a culturing process. The basal medium can be the medium that cells are inoculated into before a cell culture cycle. The basal cell culture medium can be supplied prior to a cell culture cycle for a batch or fed-batch cell culture. A basal cell culture medium may also be supplied as a feed medium, continuously or in discreet increments, to the cell culture during the culturing process, with or without period cell and/or product harvest before termination of the culture (i.e., fed-batch cell culture).

As used herein, “feed medium” refers to cell culture medium containing cell culture nutrients supplied to a culturing vessel as a feed medium, continuously or in discreet increments, to the cell culture during the culturing process, with or without period cell and/or product harvest before termination of the culture (i.e., fed-batch cell culture).

“Batch culture” as used herein refers to a method of culturing cells in which no additional components are provided to the culture at a time or times subsequent to the beginning of the culture process, i.e. all components for cell culturing (including the cells and all culture nutrients and components) are supplied to the culturing vessel at the start of the culturing process.

“Fed-batch culture” as used herein refers to a method of culturing cells in which additional components are provided to the culture at a time or times subsequent to the beginning of the culture process. Thus, it is a batch culture wherein the cells and culture medium are supplied to the culturing vessel initially, and additional culture nutrients are fed to the culture during the culturing process, with or without periodic cell and/or product harvest before termination of culture. If the feeding is continuous the process is called “continuous fed batch” or if the feeding is in discrete increments the process is called “fed batch bolus”. The process can be guided by additional “surrogate” markers (e.g. cell number, specific metabolite or substrate, on-line analytic signal) using distinct settings or algorithms. A fed-batch culture is typically stopped at some point and the cells and/or components in the medium are harvested and optionally purified.

The term “titer” as used herein refers to the total amount of an expressed protein product produced by a cell culture divided by a given amount of medium volume. Titer can be expressed or assessed in terms of a relative measurement, such as a percentage increase in titer as compared obtaining the protein product under different culture conditions.

### The Method as Reported Herein

Herein is reported a method for the production of an immunoconjugate by a specific way of culturing the cells that enclose the nucleic acids that encode the immunoconjugate.

It has been found by the inventors that it is possible to produce an immunoconjugate of interest with reduced levels of product related impurities as well as process related impurities.

Different exemplary cultivation processes have been investigated and compared.

| | Inoculation | | | |
|---|---|---|---|---|
| | cell density | | | Process |
| | (E5cells/mL) | pH value | Temperature | length |
| Process 1 | 3.5 | 7.0 | 37 (day 1 to 14) | 14 days |
| (Standard) | | | | |
| Process 2 | 6.5 | 7.0 | 37 (day 1-7) | 13 days |
| | | | 34 (day 8-13) | |
| Process 3 | 9.0 | 7.0 (day 1-7) | 37 (day 1-7) | 14 days |
| | | 7.3 (day 8-14) | 34 (day 8-14) | |

In more detail, it has been found that the levels of certain impurities during and at the end of the cultivation period/fermentation can be reduced if a shift of the temperature and the pH value is performed during the cultivation. Specifically, the temperature is reduced and the pH value is increased. Impurities that can be reduced are for example process related impurities like host cell proteins (e.g. Phospholipase B-like 2 or Clusterin) or product related impurities such as, e.g. aggregates or fragments of the immunoconjugates. For example, if the immunoconjugate comprises an interleukin like IL-2 as effector moiety, the IL-2 is prone to cleavage and therefore fragments of the immunoconjugate accumulate. Higher levels of impurities lead e.g. to contaminated products and/or laborious and complicated efforts for impurity removal. The shift of the cultivation conditions does not lead to other severe disadvantages. It could be shown that the change in cultivation conditions result in comparable levels of viable cell density and cell viability.

Accordingly, one aspect as reported herein is a method for producing an immunoconjugate with reduced product- and process-related impurities by culturing mammalian cells that contain one or more nucleic acids encoding the immunoconjugate of interest in a cell culture medium, wherein the one or more nucleic acids are expressed under the conditions of cell culture comprising the steps of:
culturing the mammalian cells in a cell culture medium at a first temperature and at a first pH;
reducing the first temperature of the cell culture medium to a second temperature; and increasing the first pH of the cell culture medium to a second pH;
recovering the immunoconjugate from the cells or the cell culture medium,

and thereby producing the immunoconjugate.

If necessary, the produced immunoconjugate can be further purified by standard purification methods.

In one embodiment the method further comprises purifying the immunoconjugate with one or more purification steps.

In one embodiment the reduction to the second temperature and the increase to a second pH is (performed) after 2 to 9 days of a total cultivation time of 13 to 14 days. In one embodiment the reduction to the second temperature and the increase to a second pH is (performed) after 7 to 9 days of a total cultivation time of 13 to 14 days. In one embodiment the reduction to the second temperature and the increase to a second pH is (performed) after about half of the culturing time (i.e. the shift is performed at the transition from the growth phase to the production phase). In one embodiment the reduction to the second temperature and the increase to a second pH is (performed) after about 8 days of a total cultivation time of 13 to 14 days.

In one embodiment the reduction to the second temperature and the increase to a second pH is (performed) at the same time. In one embodiment the reduction to the second temperature and the increase to a second pH is (performed) at different time points during a period of about 6 hours. In one embodiment first the reduction to the second temperature is performed and subsequently the increase to a second pH is performed.

It has been found that the effect of impurity reduction is pronounced if the temperature is reduced to certain temperature ranges. This is without leading e.g. to significant reduction of product titer. Contrarily, net product titers are kept at comparable levels.

In one embodiment the first temperature is 37° C.+/−0.5° C. and the second temperature is in the range of 28° C. to 34° C. (i.e. the first temperature is reduced by 3° C. to 9° C.). In one embodiment the first temperature is 37° C.+/−0.5° C. and the second temperature is in the range of 33° C. to 34° C. (i.e. is reduced by 3° C. to 4° C.). In one embodiment the first temperature is 37° C.+/−0.5° C. and the second temperature is in the range of 29° C. to 30° C. (i.e. is reduced by 7° C. to 8° C.). In one embodiment the first temperature is 37° C.+/−0.5° C. and is reduced to a second temperature of about 34° C.+/−0.5° C. In one embodiment the first temperature is 37° C.+/−0.5° C. and is reduced to a second temperature of about 29° C.+/−0.5° C.

It has been found, that in addition to reducing the temperature, also the pH value is increased to certain values for optimal results.

In one embodiment the second pH is 7.25 or higher. In one embodiment the first pH is pH 7+/−0.05 pH units and the first pH is increased by 0.25 to 0.4 pH units to the second pH. In one embodiment the first pH is 7+/−0.05 pH units and is increased by 0.3 pH units to the second pH. In one embodiment the first pH is 7+/−0.05 pH units and is increased to the second pH of about 7.3+/−0.05 pH units. In one embodiment the first pH is pH 7+/−0.1 pH units and the first pH is increased by 0.25 to 0.4 pH units to the second pH. In one embodiment the first pH is 7+/−0.1 pH units and is increased by 0.3 pH units to the second pH. In one embodiment the first pH is 7+/−0.1 pH units and is increased to the second pH of about 7.3+/−0.1 pH units.

Also the adjustment of the inoculation cell density can further improve the desired results of low impurity levels, while maintaining product titers.

In one embodiment the inoculation cell density is from about 600000 (6×10⁵) to about 1000000 (1×10⁶) cells/ml. In one embodiment the inoculation cell density is about 600000 (6×10⁵) or about 1000000 (1×10⁶) cells/ml. In one embodiment the inoculation cell density is about 650000 (6.5×10⁵). In one embodiment the inoculation cell density is about 900000 (9×10⁵). In one embodiment the inoculation cell density is increased by a factor of 2 to 4, preferably about 3, compared to a cultivation without temperature and pH shift.

In one embodiment the osmolality of the cell culture medium is 300 mOsmol/kg+/−15 mOsmol/kg or more. In one embodiment the osmolality of the cell culture medium is 350 mOsmol/kg+/−15 mOsmol/kg or more. In one embodiment the osmolality of the cell culture medium is 400 mOsmol/kg+/−15 mOsmol/kg or more. In one embodiment the osmolality of the cell culture medium is from about 250 mOsmol/kg to about 800 mOsmol/kg. In one embodiment the osmolality of the cell culture medium is from about 300 mOsmol/kg to about 600 mOsmol/kg. In one embodiment the osmolality of the cell culture medium is about 400 mOsmol/kg. In one embodiment the osmolality of the cell culture medium is from 350 mOsmol/kg+/−15 mOsmol/kg to 800 mOsmol/kg+/−15 mOsmol/kg.

In one embodiment the immunoconjugate comprises IL-2 or a variant thereof (i.e. the immunoconjugate is an IL-2 based immunoconjugate). In one embodiment the immunoconjugate is a targeted immunoconjugate. In one embodiment the immunoconjugate is a CEA-targeted IL-2 immunoconjugate, a FAP-targeted IL-2 immunoconjugate or an IgG-IL-2 immunoconjugate or a FAP-targeted 4-1-BBL immunoconjugate. In one embodiment the immunoconjugate is a CEA-targeted IL-2 immunoconjugate or an IgG-IL-2 immunoconjugate or a FAP-targeted 4-1-BBL immunoconjugate.

In one embodiment the immunoconjugate is a CEA-targeted IL-2 immunoconjugate. In one embodiment the immunoconjugate is a CEA-targeted IL-2 immunoconjugate that comprises the amino acid sequences of SEQ ID NO: 01 to SEQ ID NO: 03. In one embodiment the immunoconjugate is a CEA-targeted IL-2 immunoconjugate that comprises the amino acid sequences of SEQ ID NO: 04 to SEQ ID NO: 06.

In one embodiment the immunoconjugate is a FAP-targeted IL-2 immunoconjugate. In one embodiment the immunoconjugate is a FAP-targeted IL-2 immunoconjugate that comprises the amino acid sequences of SEQ ID NO: 07, SEQ ID NO: 08 and SEQ ID NO: 03. In one embodiment the immunoconjugate is a FAP-targeted IL-2 immunoconjugate that comprises the amino acid sequences of SEQ ID NO: 09 to SEQ ID NO: 11.

In one embodiment the immunoconjugate is an IgG-IL-2 immunoconjugate. In one embodiment the immunoconjugate is an IgG-IL-2 immunoconjugate that comprises the amino acid sequences of SEQ ID NO: 12 to SEQ ID NO: 14. In one embodiment the immunoconjugate is an IgG-IL-2 immunoconjugate that comprises the amino acid sequences of SEQ ID NO: 15 to SEQ ID NO: 16.

In one embodiment the immunoconjugate is a FAP-targeted 4-1-BBL immunoconjugate (as described in WO 2016/075278).

In one embodiment the product- and process-related impurities are selected from the group comprising host cell proteins, Clusterin, PLBL2, aggregates or fragments of the immunoconjugates.

In one embodiment the product-related impurities are selected from the group comprising aggregates or fragments of the immunoconjugates. In one embodiment the product-related impurities are fragments of the immunoconjugates. In one embodiment the product-related impurities are fragmented IL-2.

In one embodiment the process-related impurities are selected from the group comprising host cell proteins, Clusterin or PLBL2.

One aspect as reported herein is an immunoconjugate obtainable with the method as reported herein.

In one embodiment the immunoconjugate obtainable with the method as reported herein is a CEA-targeted IL-2 immunoconjugate, a FAP-targeted IL-2 immunoconjugate or an IgG-IL-2 immunoconjugate or a FAP-targeted 4-1-BBL immunoconjugate.

In one embodiment the immunoconjugate obtainable with the method as reported herein is a CEA-targeted IL-2 immunoconjugate. In one embodiment the immunoconjugate obtainable with the method as reported herein is a CEA-targeted IL-2 immunoconjugate that comprises the amino acid sequences of SEQ ID NO: 01 to SEQ ID NO: 03. In one embodiment the immunoconjugate obtainable with the method as reported herein is a CEA-targeted IL-2 immunoconjugate that comprises the amino acid sequences of SEQ ID NO: 04 to SEQ ID NO: 06.

In one embodiment the immunoconjugate obtainable with the method as reported herein is a FAP-targeted IL-2 immunoconjugate. In one embodiment the immunoconjugate obtainable with the method as reported herein is a FAP-targeted IL-2 immunoconjugate that comprises the amino acid sequences of SEQ ID NO: 07, SEQ ID NO: 08 and SEQ ID NO: 03. In one embodiment the immunoconjugate obtainable with the method as reported herein is a FAP-targeted IL-2 immunoconjugate that comprises the amino acid sequences of SEQ ID NO: 09 to SEQ ID NO: 11.

In one embodiment the immunoconjugate obtainable with the method as reported herein is an IgG-IL-2 immunoconjugate. In one embodiment the immunoconjugate obtainable with the method as reported herein is an IgG-IL-2 immunoconjugate that comprises the amino acid sequences of SEQ ID NO: 12 to SEQ ID NO: 14. In one embodiment the immunoconjugate obtainable with the method as reported herein is an IgG-IL-2 immunoconjugate that comprises the amino acid sequences of SEQ ID NO: 15 to SEQ ID NO: 16.

In one embodiment the immunoconjugate obtainable with the method as reported herein is a FAP-targeted 4-1-BBL immunoconjugate (as described in WO 2016/075278).

The invention also provides for compositions comprising immunoconjugates with reduced levels of impurities. For example, the compositions comprise only low levels of fragmented IL-2, i.e. molecules that have only part of the genetically anticipated full protein sequence. Such molecules with fragmented effector moiety/IL-2 can for example be detected as heavy chains containing IL-2 with lower molecular weight.

One aspect as reported herein is a composition comprising an immunoconjugate and a reduced level of fragments/fragmented effector moiety (e.g. IL-2), aggregates and host cell proteins.

One aspect as reported herein is a composition comprising a CEA-targeted IL-2 immunoconjugate and a reduced level of fragmented IL-2, aggregates and host cell proteins.

One aspect as reported herein is a FAP-targeted IL-2 immunoconjugate and a reduced level of fragmented IL-2, aggregates and host cell proteins.

One aspect as reported herein is an IgG-IL-2 immunoconjugate and a reduced level of fragmented IL-2, aggregates and host cell proteins.

One aspect as reported herein is a FAP-targeted 4-1-BBL immunoconjugate and a reduced level of fragmented 4-1-BBL, aggregates and host cell proteins.

One aspect as reported herein is a composition comprising a CEA-targeted IL-2 immunoconjugate and a reduced level of fragmented IL-2, aggregates and host cell proteins obtainable by the method according to claim 1.

One aspect as reported herein is a composition comprising a FAP-targeted IL-2 immunoconjugate and a reduced level of fragmented IL-2, aggregates and host cell proteins obtainable by the method according to claim 1.

One aspect as reported herein is a composition comprising an IgG-IL-2 immunoconjugate and a reduced level of fragmented IL-2, aggregates and host cell proteins obtainable by the method according to claim 1.

One aspect as reported herein is a composition comprising a FAP-targeted 4-1-BBL immunoconjugate and a reduced level of fragmented 4-1-BBL, aggregates and host cell proteins obtainable by the method according to claim 1.

In one embodiment the (relative) amount of fragmented IL-2 is 10% or less. In one embodiment the (relative) amount of fragmented IL-2 is 9% or less. In one embodiment the (relative) amount of fragmented IL-2 is 8% or less. In one embodiment the (relative) amount of fragmented IL-2 is 7% or less. In one embodiment the (relative) amount of fragmented IL-2 is 6% or less. In one embodiment the (relative) amount of fragmented IL-2 is 5% or less. In one embodiment the (relative) amount of fragmented IL-2 is 4% or less.

In one embodiment the (relative) amount of aggregates is 5% or less. In one embodiment the (relative) amount of aggregates is 4% or less. In one embodiment the (relative) amount of aggregates is 3% or less. In one embodiment the (relative) amount of aggregates is 2% or less. In one embodiment the (relative) amount of aggregates is 1% or less.

In one embodiment the (relative) amount of Clusterin is 30000 ng/ml or less. In one embodiment the (relative) amount of Clusterin is 25000 ng/ml or less. In one embodiment the (relative) amount of Clusterin is 20000 ng/ml or less. In one embodiment the (relative) amount of Clusterin is 15000 ng/ml or less.

In one embodiment the ratio of Clusterin to product titer is 15 ng/μg or less. In one embodiment the ratio of Clusterin to product titer is 13 ng/μg or less. In one embodiment the ratio of Clusterin to product titer is 10 ng/μg or less.

In one embodiment the (relative) amount of PLBL2 is 2000 ng/ml or less. In one embodiment the (relative) amount of PLBL2 is 1500 ng/ml or less. In one embodiment the (relative) amount of PLBL2 is 1200 ng/ml or less. In one embodiment the (relative) amount of PLBL2 is 1000 ng/ml or less.

In one embodiment the ratio of PLBL2 to product titer is 1.5 ng/μg or less. In one embodiment the ratio of PLBL2 to product titer is 1.2 ng/μg or less. In one embodiment the ratio of PLBL2 to product titer is 1 ng/μg or less.

In one embodiment the (relative) amount of total host cell protein is between 500000 and 2500000 ng/ml.

In one embodiment the ratio of total host cell protein to product titer is 1200 ng/μg or less. In one embodiment the ratio of total host cell protein to product titer is 1000 ng/μg or less. In one embodiment the ratio of total host cell protein to product titer is 800 ng/μg or less.

In one embodiment the cell culture is a large-scale cell culture. In one embodiment the cell culture has a volume of about 100 liters to about 15000 liters. In one embodiment the cell culture has a volume of about 1000 liters to about 15000 liters.

In one embodiment the cell culture is a batch cell culture.

In one embodiment the cell culture is a fed-batch cell culture.

In one embodiment the cell culture is a fed-batch cell culture with bolus feeding.

In one embodiment the cell culture is a fed-batch cell culture with continuous feeding.

In one embodiment the medium is a serum free medium. In one embodiment the medium is a protein-free medium. In one embodiment the medium is a chemically-defined medium. In one embodiment the medium is a protein-free chemically defined medium.

In one embodiment the mammalian cell is a CHO cell. In one embodiment the mammalian cell is a CHO K1 cell.

One aspect as reported herein is a method for producing a CEA-targeted IL-2 immunoconjugate, or a FAP-targeted IL-2 immunoconjugate or an IgG-IL-2 immunoconjugate or a FAP-targeted 4-1-BBL immunoconjugate with reduced product- and process-related impurities by culturing CHO cells that contain one or more nucleic acids encoding the immunoconjugate of interest in a cell culture medium, wherein the one or more nucleic acids are expressed under the conditions of cell culture comprising the steps of:
culturing the mammalian cells in a cell culture medium at a first temperature and at a first pH;
reducing the first temperature of the cell culture medium to a second temperature; and increasing the first pH of the cell culture medium to a second pH;
recovering the immunoconjugate from the cells or the cell culture medium,

and thereby producing the immunoconjugate.

One aspect as reported herein is a method for producing an IL-2 immunoconjugate with reduced product- and process-related impurities by culturing CHO cells that contain one or more nucleic acids encoding the immunoconjugate of interest in a cell culture medium, wherein the one or more nucleic acids are expressed under the conditions of cell culture comprising the steps of:
culturing the mammalian cells in a cell culture medium at a first temperature and at a first pH;
reducing the first temperature of the cell culture medium to a second temperature; and increasing the first pH of the cell culture medium to a second pH;
recovering the immunoconjugate from the cells or the cell culture medium,

and thereby producing the immunoconjugate.

One aspect as reported herein is a method for producing a CEA-targeted IL-2 immunoconjugate or an IgG-IL-2 immunoconjugate with reduced product- and process-related impurities by culturing CHO cells that contain one or more nucleic acids encoding the immunoconjugate of interest in a cell culture medium, wherein the one or more nucleic acids are expressed under the conditions of cell culture comprising the steps of:
culturing the mammalian cells in a cell culture medium at a first temperature and at a first pH;
reducing the first temperature of the cell culture medium to a second temperature; and increasing the first pH of the cell culture medium to a second pH;
recovering the immunoconjugate from the cells or the cell culture medium,

and thereby producing the immunoconjugate.

One aspect as reported herein is a method for producing a CEA-targeted IL-2 immunoconjugate or an IgG-IL-2 immunoconjugate with reduced levels of fragments (of the immunoconjugate) (and/) or aggregates by culturing mammalian cells that contain one or more nucleic acids encoding the immunoconjugate of interest in a cell culture medium, wherein the one or more nucleic acids are expressed under the conditions of cell culture comprising the steps of:
culturing the mammalian cells in a cell culture medium at a first temperature and at a first pH;
reducing the first temperature of the cell culture medium to a second temperature; and increasing the first pH of the cell culture medium to a second pH;
recovering the immunoconjugate from the cells or the cell culture medium,

and thereby producing the immunoconjugate.

The following examples, figures and sequences are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 Different process variants tested. The different key parameters for inoculation cell density, pH shift, temperature shift, and process length are shown for process 1 (reference/standard process), process 2 and process 3.

FIG. 2 Process variants 2 and 3 reduce supernatant Clusterin concentration at harvest for a clone expressing cM1. Cell-free samples of three fed-batch processes lasting 13 (Process 2) and 14 days (Process 3) in 2 L glass bioreactors using a recombinant CHO cell line expressing a complex cytokine-IgG fusion protein (cM1=CEA-IL-2 immunoconjugate) were analyzed for (A) Clusterin, (B) net titer, and (C) the ratio of Clusterin per net titer. Whiskers represent respective standard deviation (n=3)

FIG. 3a Process variants 2 and 3 reduce supernatant Clusterin concentration at harvest for two clones expressing cM2. Cell-free samples of a fed-batch processes lasting 13 (Process 2) and 14 days (Process 3) in 2 L glass bioreactors using two recombinant CHO cell lines (Clone 1 and Clone 2) expressing a complex cytokine-IgG fusion protein (cM2=IgG-IL-2 immunoconjugate) were analyzed for (A) Clusterin, (B) titer, and (C) the ratio of Clusterin per titer.

FIG. 3b Process variants 2 and 3 reduce supernatant CHO host cell protein (HCP) concentration at harvest for two clones expressing cM2. Cell-free samples of a fed-batch processes lasting 13 (Process 2) and 14 days (Process 3) in 2 L glass bioreactors using two recombinant CHO cell lines (Clone 1 and Clone 2) expressing a complex cytokine-IgG fusion protein (cM2=IgG-IL2 immunoconjugate) were analyzed for (A) CHO host cell protein and (B) the ratio of CHO HCP per titer.

FIG. 3c Process variants 2 and 3 reduce supernatant PLBL2 concentration at harvest for two clones expressing cM2. Cell-free samples of a fed-batch processes lasting 13 (Process 2) and 14 days (Process 3) in 2 L glass bioreactors using two recombinant CHO cell lines (Clone 1 and Clone 2) expressing a complex cytokine-IgG fusion protein (cM2=IgG-IL2 immunoconjugate) were analyzed for (A) PLBL2 and (B) the ratio of PLBL2 per titer.

FIG. 4 Process variant 3 reduces supernatant Clusterin concentration at harvest in production scale/large scale. Cell-free samples of a 13 day (Process 2) and 14 day (Process 3) fed-batch processes in 250 L single use bioreactors using a recombinant CHO cell line expressing a complex cytokine-IgG fusion protein (cM2=IgG-IL-2 immunoconjugate) were analyzed for Clusterin.

FIG. 5 Reduction of cM2 fragments and aggregates by process 3. (A) Fragments: heavy chain fragments+non-glycosylated heavy chain, measured by CE-SDS, and (B) Aggregates (HMW species), measured by SEC are shown for clone 1 and clone 2 expressing cM2. All experiments were performed in 2 L glass bioreactors.

FIG. 6 Modulation of supernatant Clusterin levels by process pH. Cell-free samples of a 14 day fed-batch process (Process 1) with pH shifts to 6.8, 7.0, 7.2, or 7.4 at day 8 in 2 L glass bioreactors using two recombinant CHO cell lines, clone 6 and clone 7, expressing a complex IgG fusion protein (cM3=FAP-4-1-BBL immunoconjugate) were analyzed for Clusterin.

FIG. 7 Modulation of supernatant Clusterin levels by process temperature. Cell-free samples of a 14 day fed-batch process (Process 1) with temperature shift to 33° C. or no temperature shift (37° C.) at day 8 in shake flasks using two recombinant CHO cell lines, clone 6 and clone 7, expressing a complex IgG fusion protein (cM3=FAP-4-1-BBL immunoconjugate) were analyzed for (A) Clusterin and (B) product titer. Whiskers represent respective standard deviation.

FIG. 8 Modulation of supernatant Clusterin levels by process osmolality. Cell-free samples of a 14 day fed-batch process (Process 1) with different base media osmolalities (300 and 400 mOsmol/kg) in shake flasks using the recombinant CHO cell lines clone 6 expressing a complex IgG fusion protein (cM3=FAP-4-1-BBL immunoconjugate) were analyzed for (A) Clusterin, (B) product titer, and (C) the ratio Clusterin per titer. Whiskers represent respective standard deviation.

FIG. 9 Reduction of cM1 (=CEA-IL-2 immunoconjugate) fragments and aggregates. (A) Fragments: heavy chain fragments+non-glycosylated heavy chain, measured by CE-SDS, and (B) Aggregates (HMW species), measured by SEC are shown for a clone expressing cM1. All experiments were performed in in 2 L glass bioreactors.

FIG. 10 Net titers for Processes 1, 2 and 3. Net titers for the different processes were determined by subtracting the amount of fragmented products from the total product titer. All processes show comparable levels of net titer.

FIG. 11 Viable cell density (A) and cell viability (B) is shown. Fed-batch processes lasting 13 (Process 2, grey markers and lines) and 14 days (Process 3, black markers and lines) in 2 L glass bioreactors using two recombinant CHO cell lines (Clone 1, circle markers, and Clone 2, square markers) expressing a complex cytokine-IgG fusion protein (cM2=IgG-IL2 immunoconjugate) were analyzed for (A) viable cell density and (B) cell viability.

FIG. 12 Viable cell density (A) and cell viability (B) is shown. Fed-batch processes (n=3) lasting 14 days (process variant 1, black; process variant 2, light grey; process variant 3, dark grey) in 2 L glass bioreactors using one recombinant CHO cell line expressing a complex cytokine-IgG fusion protein (cM1=CEA-IL-2 immunoconjugate) were analyzed for (A) viable cell density and (B) cell viability. All processes show comparable cell viability and viable cell densities.

### DESCRIPTION OF THE SEQUENCE LISTING

| | |
|---|---|
| SEQ ID NO: 01 | variable heavy chain domain VH of anti-CEA IgG |
| | comprised in CEA-targeted IL-2 immunoconjugate |
| SEQ ID NO: 02 | variable light chain domain VL of anti-CEA IgG |
| | comprised in CEA-targeted IL-2 immunoconjugate |
| SEQ ID NO: 03 | Interleukin 2 variant (IL-2) comprised in CEA- |
| | targeted IL-2 immunoconjugate and FAP-targeted |
| | IL-2 immunoconjugate |
| SEQ ID NO: 04 | Full heavy chain (knob) with IL-2 comprised in |
| | CEA-targeted IL-2 immunoconjugate |
| SEQ ID NO: 05 | Full heavy chain (hole) comprised in CEA-targeted |
| | IL-2 immunoconjugate |
| SEQ ID NO: 06 | Full light chain comprised in CEA-targeted IL-2 |
| | immunoconjugate |
| SEQ ID NO: 07 | variable heavy chain domain VH of anti-FAP IgG |
| | comprised in FAP-targeted IL-2 immunoconjugate |
| SEQ ID NO: 08 | variable light chain domain VL of anti-FAP IgG |
| | comprised in FAP-targeted IL-2 immunoconjugate |
| SEQ ID NO: 09 | Full heavy chain (knob) with IL-2 comprised in |
| | FAP-targeted IL-2 immunoconjugate |
| SEQ ID NO: 10 | Full heavy chain (hole) comprised in FAP-targeted |
| | IL-2 immunoconjugate |
| SEQ ID NO: 11 | Full light chain comprised in FAP-targeted IL-2 |
| | immunoconjugate |
| SEQ ID NO: 12 | variable heavy chain domain VH of untargeted IgG |
| | comprised in IgG-IL-2 immunoconjugate |
| SEQ ID NO: 13 | variable light chain domain VL of untargeted IgG |
| | comprised in IgG-IL-2 immunoconjugate |
| SEQ ID NO: 14 | Interleukin 2 variant (IL-2) comprised in IgG-IL-2 |
| | immunoconjugate |
| SEQ ID NO: 15 | Full heavy chain with IL-2 comprised in IgG-IL-2 |
| | immunoconjugate |
| SEQ ID NO: 16 | Full light chain comprised in IgG-IL-2 |
| | immunoconjugate |

### Example 1

### General Methods and Material

### Cell Lines

In the context of this work, transfected CHO (Chinese hamster ovary) K1 cell lines were used.

### Immunoconjugates

The current invention is exemplified using a number of exemplary immunoconjugates such as a CEA-targeted-IL-2 immunoconjugate (anti-CEA antibody conjugated to IL-2) as described in WO 2012/146628 or SEQ ID NO: 01 to SEQ ID NO: 06; or a FAP-targeted-IL-2 immunoconjugate as described in WO 2012/107417 or SEQ ID NO: 07 to SEQ ID NO: 11 and SEQ ID NO: 03; or an untargeted IgG-IL-2 immunoconjugate as reported in WO 2015/118016 or SEQ ID NO: 12 to SEQ ID NO: 16; or a FAP-targeted-4-1-BBL immunoconjugate as described in WO 2016/075278.

### Cell Culture Methods

Media and feeds required for cell culture were prepared and the parameters pH value, glucose concentration and osmolality adjusted according to the operating instructions of the supplier (Gibco/Life Technologies Inc.). Media and feeds were stored at 4° C. in the dark and consumed within four weeks (preculture medium, fermentation medium), three weeks (feed 1) and two weeks (feed 2), respectively. Correction agents were stored at room temperature and expended within 3 months (glucose solution; 50%), 6 months (sodium carbonate solution; IM) and 12 months (defoamer solution; Dow Corning® Antifoam C emulsion, food grade, 10%).

The methods described in the following part are adapted from standard protocols (Lindl, T. Zell-und Gewebekultur: Einfiihrung in die Grundlagen sowie ausgewihlte Methoden und Anwendungen. Heidelberg/Berlin, Spektrum Akademischer Verlage GmbH 2002) and operating instructions of the respective supplier.

### Preculture

Cells were thawed and precultured in shake flasks for two to three weeks in preculture medium. The incubation parameters were: Temperature 37° C., Humidity 80%, CO₂ 7%, Shaking frequency 210 rpm, Amplitude 5 cm. Cells were passaged every three to four days and expanded in preculture medium under selection pressure (Methotrexate) to the volume required for inoculation.

### Standard Cell Culture Process (Process 1)

Cells were cultured in a or 14-day fed-batch process with Feed 1, Feed 2, and glucose with a start cell density of 3.5·10E5 cells/ml. The feeds were started on day three and six of fermentation and were added continuously during the remaining days of fermentation in a rate of 2% (v/v) based on the start volume. If not stated otherwise, the pH value was adjusted with CO₂ and IM Na₂CO₃ within a dead band of 0.05 pH units. Antifoam solution and antibiotics were added if necessary. During the process, the parameters temperature, pH value, and pO₂ were monitored and controlled. The fermentation process was stopped after 14 days. The culture broth was harvested and sterile filtered. The bioreactors were dismantled and cleaned.

For adopted processes (process 2 and 3) see below.

### Fermentation Setup in the Quad System

The 2 L double wall glass bioreactors are closed with a steel lid, where built-in components such as stirrer, probes, feed ports etc. are integrated. Four vessels are controlled by one BIOSTAT® DCU3/4 (Sartorius Stedim Biotech AG) control system and, hence, termed Quad system.

The bioreactors were assembled and filled with KH₂PO₄ solution (1.4 g/l). The pH probes were calibrated and together with the pO₂ probes integrated into the bioreactor. Subsequently the system was tested for pressure tightness. The bioreactors were autoclaved (wet program: 121° C. and 1.2 bar above atmospheric pressure for 30 min) and were connected to the supply towers (DCU3 and 4, respectively). KH₂PO₄ was replaced by 900 ml fermentation medium on the day before inoculation. In addition, pH probes were calibrated, base and glucose flasks were connected to the bioreactor, and controlling (stirrer, gassing for pO₂ calibration, temperature, and pressure) was started. Before inoculation, pH probes were recalibrated, pO₂ probes were calibrated and parameters were set to fermentation conditions (Temperature 37° C., pO₂ 35%, pH 7, Pressure 100 mbar). The feedback control system for pO₂ was regulated over the following cascade: N2/air, air/O₂, and stirrer.

### Cell Growth Measurements

Viable Cell Density and Viability were assessed during the culture processes with trypan blue staining by a Cedex HiRes (Roche Diagnostics GmbH) device.

### Protein a Purification

Purification and CE were carried out according to the protocols of the suppliers. In general, frozen samples containing protein were thawed cautiously at 4° C. and kept on ice when possible. Freeze and thaw cycles were avoided. Fermentation samples were centrifuged (4000×g, 10-30 min, depending on the volume) and either purified and/or analyzed immediately or shock frozen in liquid nitrogen and stored at 80° C.

Affinity chromatography was used for purification of the antibodies. Protein A purification of small volumes (150 μl-3 ml) was performed using PureSpeed tips (Mettler Toledo).

For larger volumes (2 L-3 L) Protein A MabSelectSure columns were equilibrated. Subsequently, the fermentation supernatant was loaded for the capture step. The column was washed before eluting the protein. Finally, the pH of the eluate was adjusted to 5.0 with neutralization buffer.

### Capillary Electrophoresis (CE-SDS)

CE on a chip was performed with the Agilent Bioanalyzer 2100 or the PerkinElmer LabChip GX and the respective protein kits according to the instructions of the manufacturer.

Fragmentation was analyzed with CE in reduced state. The standard CE diagrams show different marker peaks and three product peaks: the light chain peak is visible at a size of approximately 28 kDa, the heavy chain hole (HChole, HC1) peak at about 58 kDa and the peak of the heavy chain knob (HCknob, HC2) with the attached IL-2 molecule at around 74 kDa. When fragmentation of the IL-2 moiety occurs, an additional peak is visible between the HC1 and HC2 peaks.

### Net Product Titer

The product titer was measured with the Bioprofile 100 Plus. However, the device cannot differentiate between fragmented and not fragmented antibodies. For this reason, the net titer with less fragments was calculated as shown below.

Tₙₑₜ=(1−F)·T

With: Tnet=Net product titer (less fragments) [mg/l]
T=Total titer [mg/1]

### F=Fragments [%]

### Example 2

pH and Temperature Modulation Influence Clusterin Levels in CHO Fed-Batch Processes (Exemplified with cM1/cM2)

To elucidate the effect of pH and temperature on the supernatant Clusterin levels in CHO fed-batch processes three process variants, process 1, process 2, and process 3 were compared in triplicate.

TABLE
Used process parameters tested.

| | Inoculation | | | |
|---|---|---|---|---|
| | cell density | | | Process |
| | (E5cells/mL) | pH value | Temperature | length |
| Process 1 | 3.5 | 7.0 | 37 (day 1 to 14) | 14 days |
| (Standard) | | | | |
| Process 2 | 6.5 | 7.0 | 37 (day 1-7) | 13 days |
| | | | 34 (day 8-13) | |
| Process 3 | 9.0 | 7.0 (day 1-7) | 37 (day 1-7) | 14 days |
| | | 7.3 (day 8-14) | 34 (day 8-14) | |

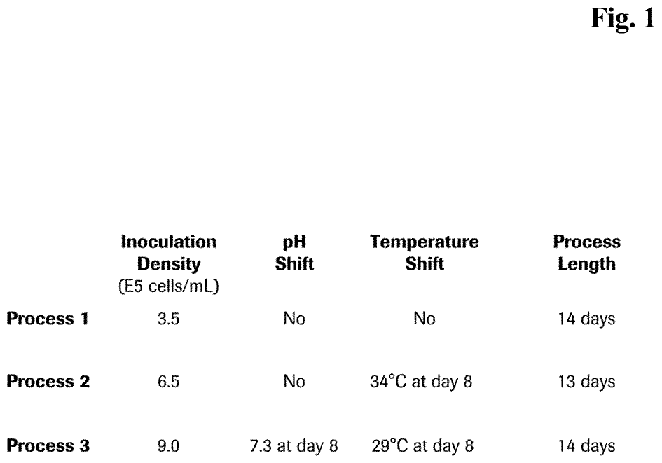

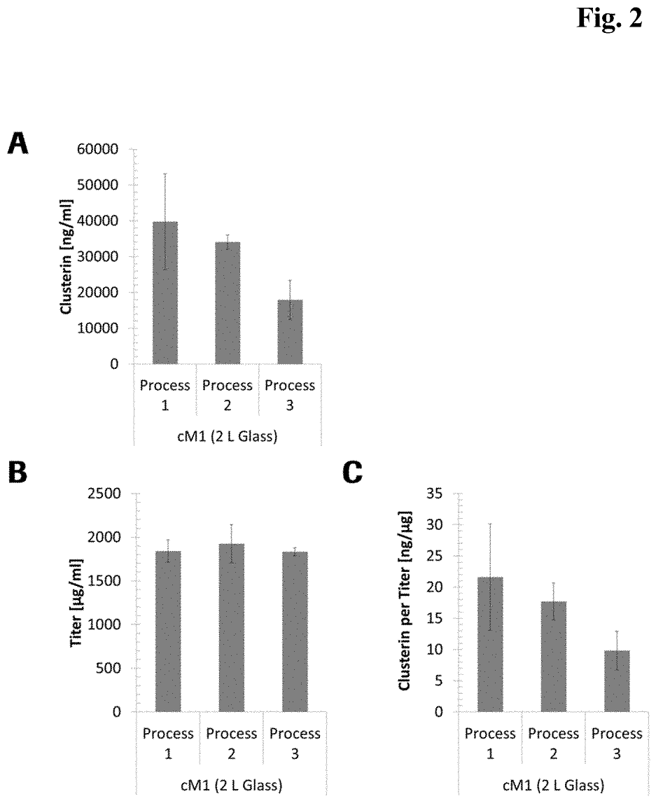

Process 2 and process 3 differ from process 1 by an elevated inoculation cell density, an additional temperature shift at day 8, for both variants and harvest at day 13 for process 2 and an additional pH shift for process 3 (FIG. 1). In the given set up, a clone stably expressing a complex cytokine-IgG fusion protein (cM1=CEA-IL-2 immunoconjugate) were used for fed-batch processes in 2 L glass bioreactors. Both, process 2 and process 3 showed reduced levels of supernatant Clusterin at harvest when compared to baseline process variant 1 (FIG. 2A). Process 2 and process 3 reached the same productivity, calculated by net product titer, as the reference process 1 (FIG. 2B). By that, the ratio of supernatant Clusterin concentration per product concentration followed the same trend as the supernatant Clusterin levels alone: Process variants 2 and 3 induce a reduced Clusterin “load” in respect for the produced molecules (FIG. 2C).

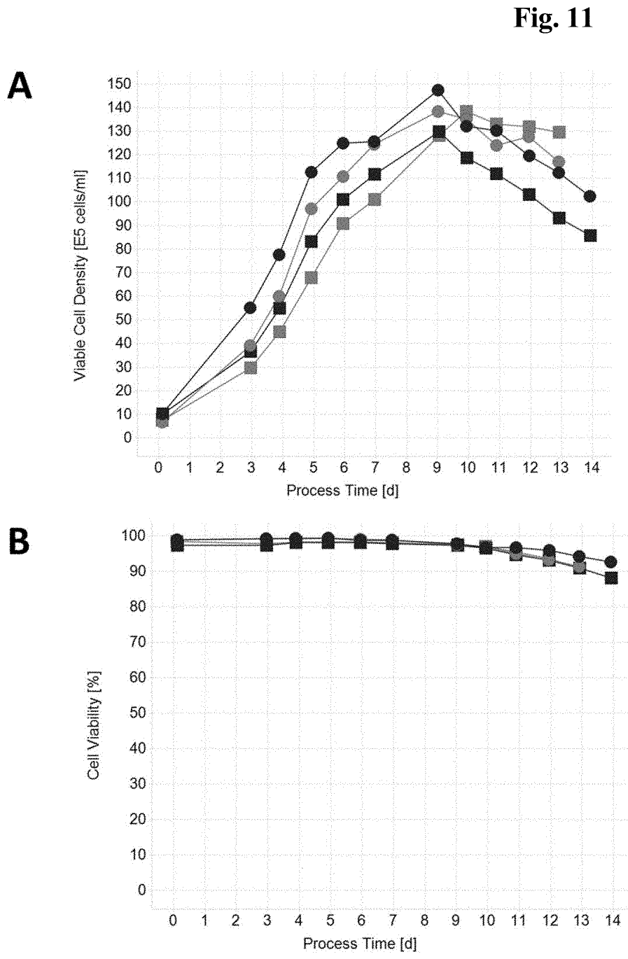

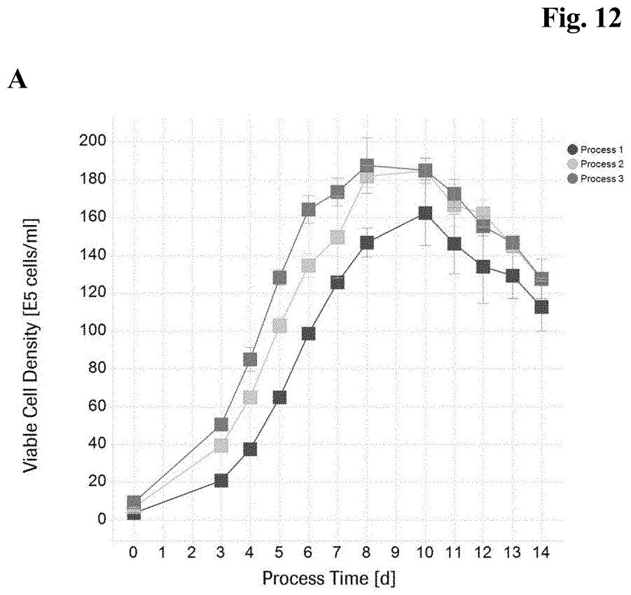

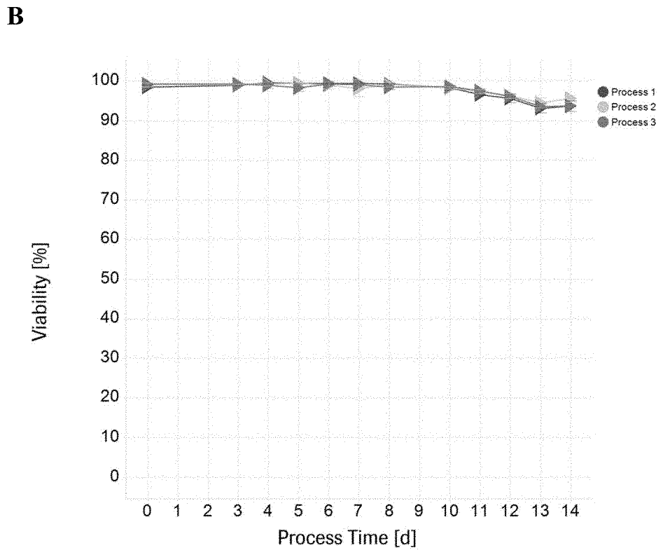

Viable cell density and cell viability were assessed and found comparable for all three processes (FIGS. 11 and 12).

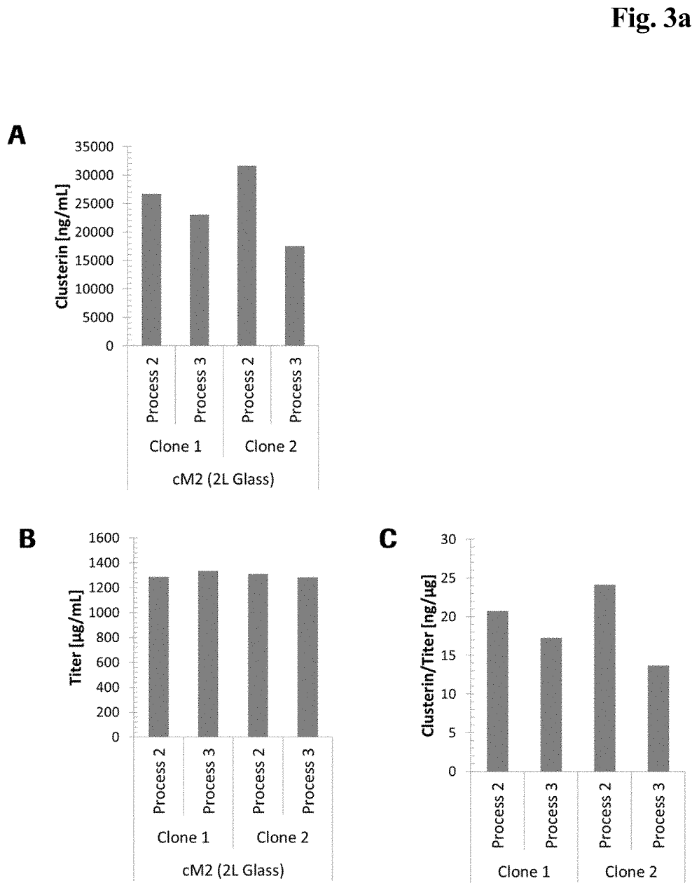

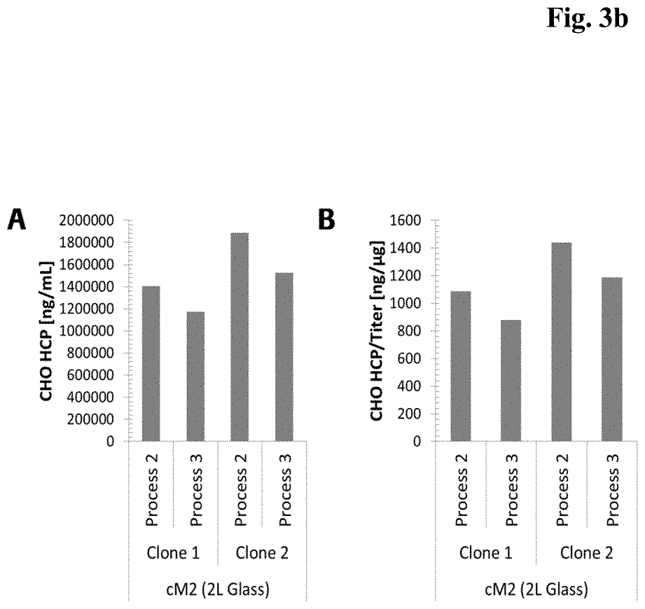

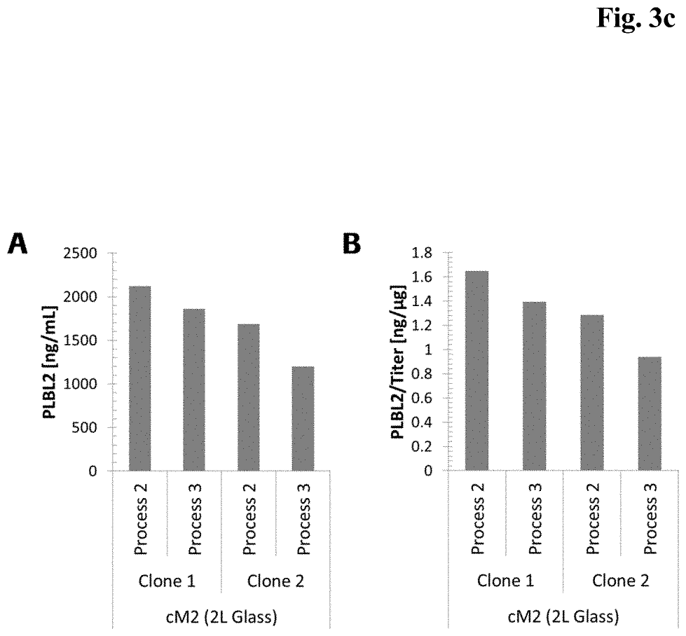

To analyze the general effect of modulating pH and temperature in CHO fed-batch processes on Clusterin levels two clones (clone 1 and clone 2) stably expressing another complex cytokine-IgG fusion protein (cM2=IgG-IL-2 immunoconjugate) and process variants 2 and 3 were used in 2 L bioreactor runs. As observed before the process variant 3 also showed for clone 1 and clone 2 a reduced level of supernatant Clusterin and Clusterin per product ratio, yet overall product titer remains the same (FIG. 3a,(A)). Same reduction trends for CHO host cell protein (FIG. 3b, (A)-(C)) and PLPL2 (FIG. 3c, (A)-(C)) could be observed for clone 1 and clone 2 using process variant 3.

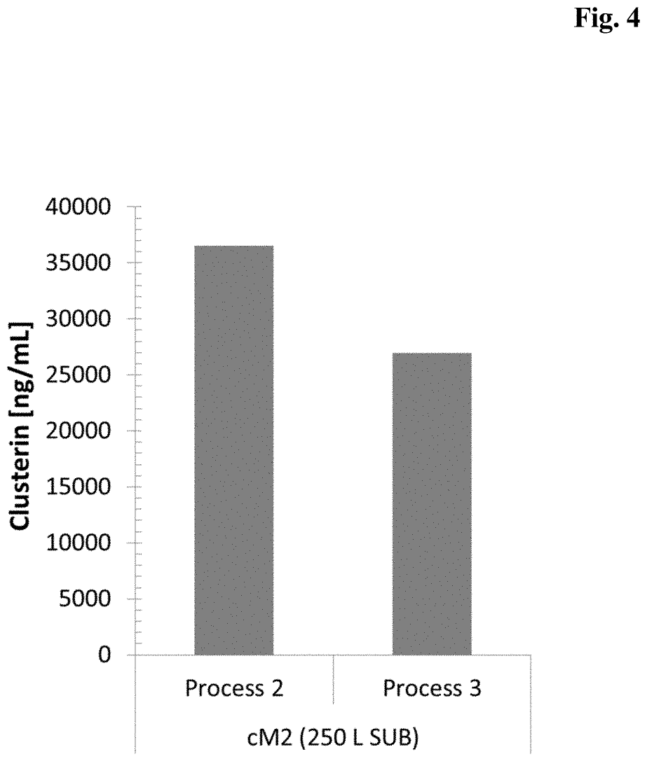

The scalability of the process variant was proven by comparing process variant 2 and 3 in fed-batch processes using 250 L single use bioreactors (SUBs) as representative production scale. Again, process variant 3 showed decreased levels of supernatant Clusterin compared to process 2 (FIG. 4).

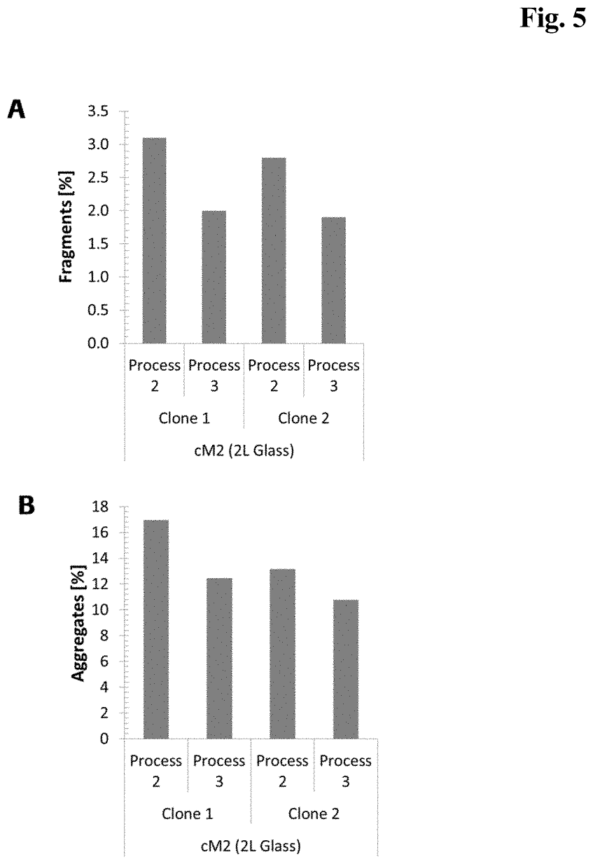

The final levels of cM2 product-related impurities (fragmentation and aggregation) at harvest, were measured by CE-SDS and SEC, respectively. Both clones tested, clone 1 and clone 2, showed a reduced level for cM2 fragmentation and aggregation when process variant 3 was used (FIG. 5).

### Example 3

Clusterin Levels can be Modulated by pH, Temperature and Medium Osmolality (Exemplified with cM3)

In fed-batch experiments using either 2 L glass bioreactors or shake flasks the process and media parameters pH, temperature and osmolality were tested to effect supernatant Clusterin levels of CHO fed-batch cultivations. For that, two clones, clone 6 and clone 7, both expressing the same complex IgG fusion protein (cM3=FAP-4-1-BBL immunoconjugate), and process variant 1 were used. Temperature and pH shifts were started at day 8 and kept until harvest at day 14. The different medium osmolalities were used from the start of the cultivation on.

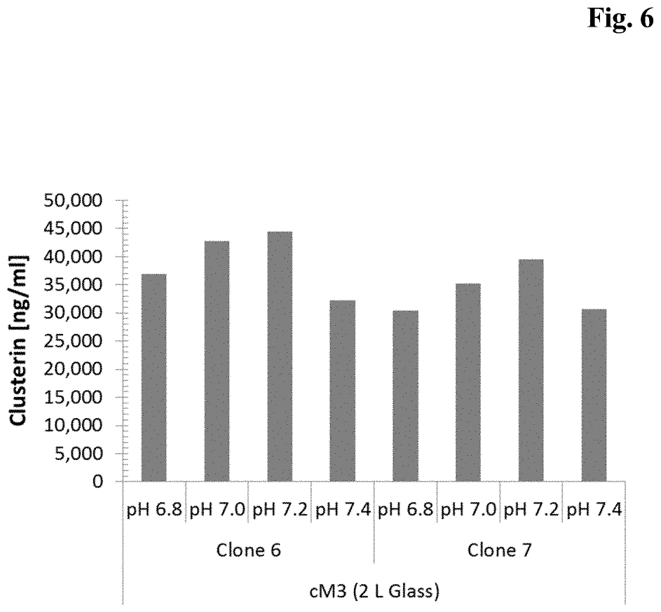

For both clones the supernatant Clusterin levels raised from pH 6.8 to pH 7.2 (FIG. 6). At pH 7.4 the Clusterin levels showed the lowest or comparable levels to 6.8 for clone 6 and clone 7, respectively.

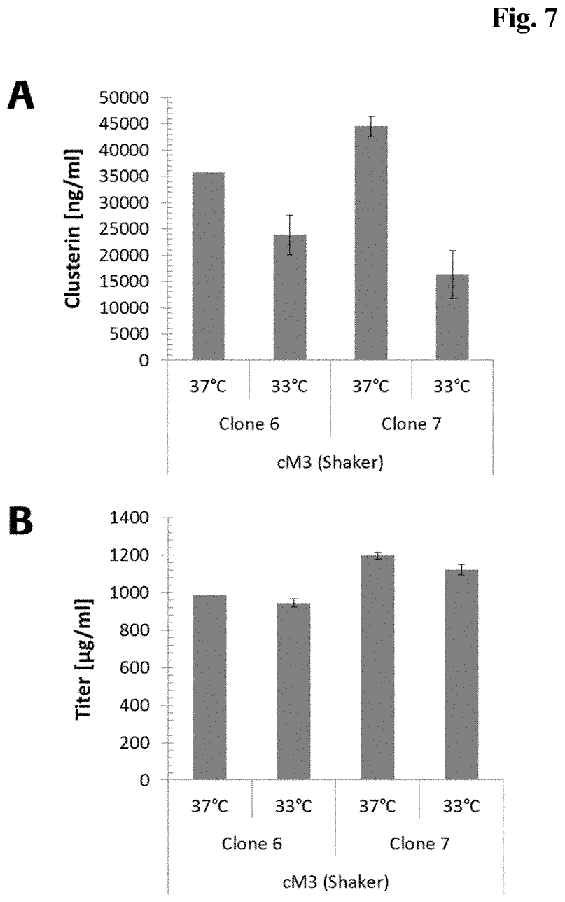

Decreasing the temperature at day 8 to 33° C. reduced the Clusterin levels by approximately 50%, yet the productivity of the cultivation remained on a comparable level (FIG. 7).

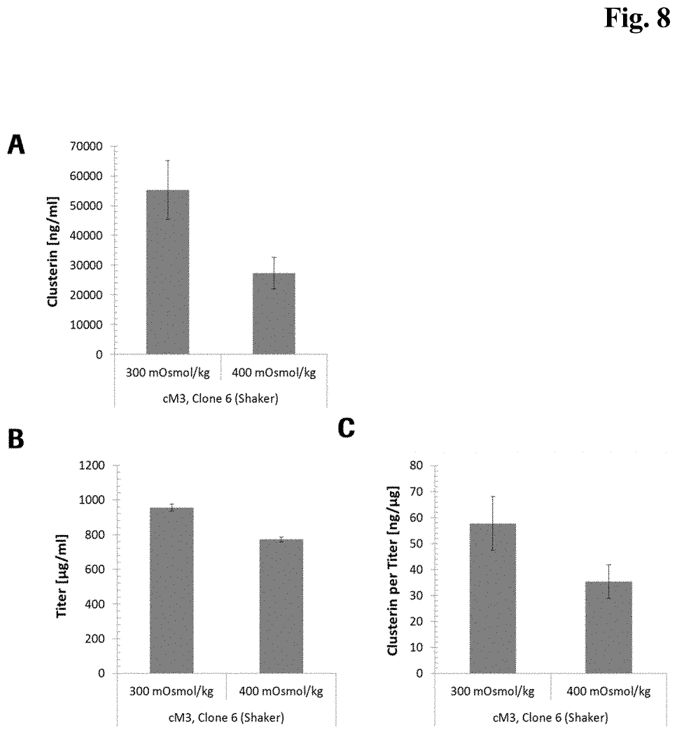

Modulation of the process osmolality for CHO cells can be used to change the cell specific productivity. In a subsequent experiment the effect of medium osmolality on supernatant Clusterin were analyzed. By using higher medium osmolality the process yielded a slight reduced final titer compared to low medium osmolality levels (FIG. 8B). However, supernatant Clusterin levels and the ratio of Clusterin per product titer were reduced by approximately 50% (FIG. 8A,C).

### Example 4

Modulation of Fragmentation and Aggregation Levels (Exemplified with cM1)

As described in Example 2 different parameters like pH and temperature on fragmentation and aggregation levels of recombinantly produced molecules in CHO cells were analyzed. Again, the three different fed-batch process variants, process 1, process 2, and process 3 (see Example 2) were compared. Here, a clone stably expressing a complex cytokine-IgG fusion protein (cM1=CEA-IL-2 immunoconjugate) were used for fed-batch processes.

The final levels of cM1 product-related impurities (fragmentation and aggregation) at harvest, were measured by CE-SDS and SEC, respectively.

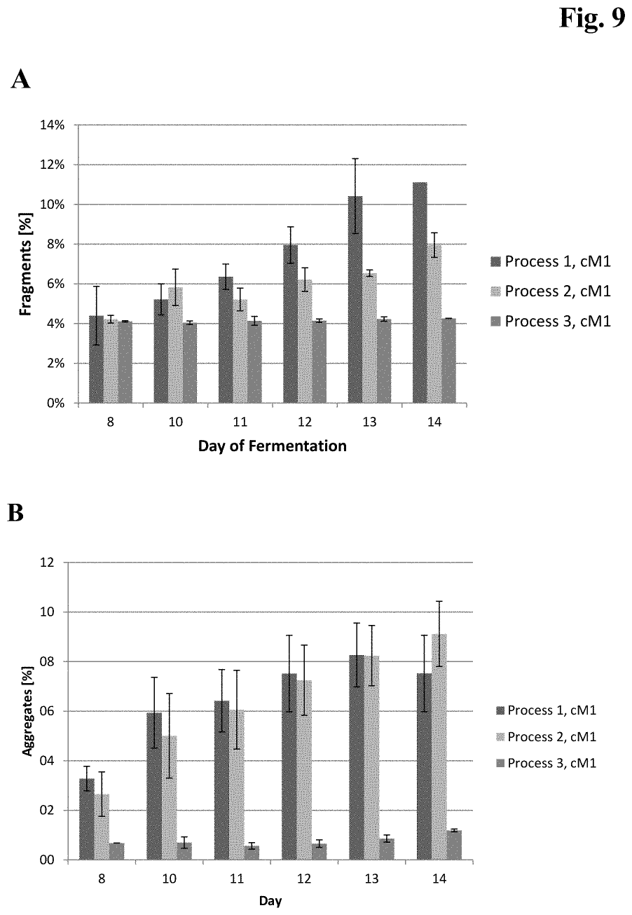

Process 2 and especially process 3 show significantly reduced levels of fragmentation (11% for process 1 vs 4% for process 3 on day 14; FIG. 9 A). Aggregate levels were significantly reduced with process 3 (FIG. 9 B).

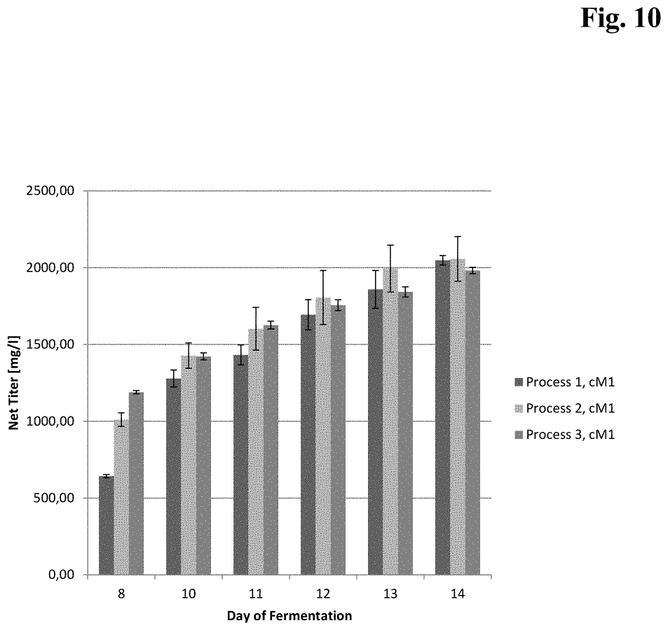

Importantly, the net titer was about the same for all three processes on day 14 (FIG. 10). Thus, productivity can be kept on a high level while fragmentation and aggregation can be reduced. Also, viable cell density and cell viability were assessed and found comparable for all three processes (FIG. 12).

### Example 5

### Purification of Immunoconjugates

Following the harvest of the produced immunoconjugates, further purification may be performed. Briefly, immunoconjugates were purified by one affinity step with protein A (HiTrap ProtA, GE Healthcare) equilibrated in 20 mM sodium phosphate, 20 mM sodium citrate pH 7.5. After loading of the supernatant, the column was first washed with 20 mM sodium phosphate, 20 mM sodium citrate, pH 7.5 and subsequently washed with 13.3 mM sodium phosphate, 20 mM sodium citrate, 500 mM sodium chloride, pH 5.45. The immunoconjugates were eluted with 20 mM sodium citrate, 100 mM sodium chloride, 100 mM glycine, pH 3. Fractions were neutralized and pooled and purified by size exclusion chromatography (HiLoad 16/60 Superdex 200, GE Healthcare) in final formulation buffer: 25 mM potassium phosphate, 125 mM sodium chloride, 100 mM glycine pH 6.7. The protein concentration of purified protein samples was determined by measuring the optical density (OD) at 280 nm, using the molar extinction coefficient calculated on the basis of the amino acid sequence. Purity and molecular weight of immunoconjugates were analyzed by SDS-PAGE in the presence and absence of a reducing agent (5 mM 1,4-dithiotreitol) and stained with Coomassie blue (SimpleBlue™ SafeStain, Invitrogen).

1. A method for producing an immunoconjugate with reduced product- and process-related impurities by culturing mammalian cells that contain one or more nucleic acids encoding the immunoconjugate of interest in a cell culture medium, wherein the one or more nucleic acids are expressed under the conditions of cell culture comprising the steps of:
culturing the mammalian cells in a cell culture medium at a first temperature and at a first pH;
reducing the first temperature of the cell culture medium to a second temperature; and
increasing the first pH of the cell culture medium to a second pH;
recovering the immunoconjugate from the cells or the cell culture medium, and thereby producing the immunoconjugate.

2. The method according to claim 1, wherein the method further comprises purifying the immunoconjugate with one or more purification steps.

3. The method according to claim 1, wherein the reduction to the second temperature and the increase to a second pH is after 2 to 9 days of a total cultivation time of 13 to 14 days.

4. The method according to claim 1, wherein both the reduction to the second temperature and the increase to the second pH is at about the same time.

5. The method according to claim 1, wherein the first temperature is 37° C.+/−0.5° C. and the second temperature is in the range of 28° C. to 34° C.

6. The method according to claim 1, wherein the first temperature is 37° C.+/−0.5° C. and is reduced to the second temperature of 29° C.+/−0.5° C.

7. The method according to claim 1, wherein the second pH is 7.25 or higher.

8. The method according to claim 1, wherein the first pH is pH 7+/−0.05 pH units and the first pH is increased by 0.25 to 0.4 pH units to the second pH.

9. The method according to claim 1, wherein the inoculation cell density is from about 600000 (6×10⁵) to about 1000000 (1×10⁶) cells/ml.

10. The method according to claim 1, wherein the osmolality of the cell culture medium is from 350 mOsmol/kg+/−15 mOsmol/kg to 800 mOsmol/kg+/−15 mOsmol/kg.

11. The method according to claim 1, wherein the immunoconjugate is a CEA-targeted IL-2 immunoconjugate, a FAP-targeted IL-2 immunoconjugate or an IgG-IL-2 immunoconjugate or a FAP-targeted 4-1-BBL immunoconjugate.

12. The method according to claim 1, wherein the cell culture is a fed-batch cell culture.

13. The method according to claim 1, wherein the mammalian cell is a CHO cell.

14. A composition comprising an immunoconjugate and a reduced level of fragments, aggregates and host cell proteins obtainable by the method according to claim 1.

15. The composition of claim 14, wherein the immunoconjugate is a CEA-targeted IL-2 immunoconjugate, a FAP-targeted IL-2 immunoconjugate or an IgG-IL-2 immunoconjugate or a FAP-targeted 4-1-BBL immunoconjugate.


SEQUENCE LISTING (16 sequences)

SEQ ID NO: 1 - protein, 121 aa, Artificial Sequence
  CH1A1A; VH
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 2 - protein, 108 aa, Artificial Sequence
  CH1A1A; VL
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 3 - protein, 133 aa, Artificial Sequence
  Quadruple mutant human IL-2 (IL-2 qm)
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 4 - protein, 598 aa, Artificial Sequence
  CH1A1A 98/99 2F1 Fab HC-Fc knob (LALA P329G)- IL-2 qm (2)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 5 - protein, 451 aa, Artificial Sequence
  CH1A1A 98/99 2F1 Fab HC-Fc hole (LALA P329G)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 6 - protein, 215 aa, Artificial Sequence
  CH1A1A 98/99 2F1 Fab LC
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 117 aa, Artificial Sequence
  4B9; VH
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 8 - protein, 108 aa, Artificial Sequence
  4B9; VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 9 - protein, 594 aa, Artificial Sequence
  4B9 Fab heavy chain-Fc (knob)-IL-2 qm
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 10 - protein, 447 aa, Artificial Sequence
  4B9 Fab heavy chain-Fc (hole)
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 215 aa, Artificial Sequence
  3F2 light chain
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 115 aa, Artificial Sequence
  DP47GS VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSS

SEQ ID NO: 13 - protein, 108 aa, Artificial Sequence
  DP47GS VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 14 - protein, 133 aa, Artificial Sequence
  IL-2 N88D T3A C125A
APASSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 15 - protein, 592 aa, Artificial Sequence
  DP47GS HC(Fc wt,P329G LALA)-IL2 N88D
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 16 - protein, 215 aa, Artificial Sequence
  DP47GS LC
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC